(19) United States
(12) Patent Application Publication
(10) Pub. No.: US 2004/0247607 A1
(43) Pub. Date: Dec. 9, 2004

(54) Use of surfactants to stabilize oocysts

(75) Inventors: Farooq Uraizee, Eureka, MO (US); Julia J. Dibner, Chesterfield, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Novus International, Inc.

(21) Appl. No.: 10/799,291

(22) Filed: Mar. 12, 2004

Related U.S. Application Data
(63) Continuation-in-part of application No. 09/708,918, filed on Nov. 8, 2000.
(60) Provisional application No. 60/163,989, filed on Nov. 8, 1999.

(51) Int. Cl. A61K 39/38; A61K 39/00; A61K 39/012

(52) U.S. Cl. 424/184.1

(57) ABSTRACT

The present invention provides compositions comprising oocysts and a surfactant capable of reducing or preventing aggregation of the oocysts. Also provided are methods for reducing or preventing oocyst aggregation in a vaccine, and methods for evaluating the ability of a surfactant to reduce or prevent oocyst aggregation.

### CROSS-REFERENCE TO RELATED APPLICATIONS

This application is a continuation-in-part of pending U.S. application Ser. No. 09/708,918, filed Nov. 8, 2000, which claims the benefit of U.S. Provisional Application No. 60/163,989, filed Nov. 8, 1999, both of which are hereby incorporated by reference in their entirety.

### BACKGROUND OF THE INVENTION

Coccidiosis is a disease of various animals in which the intestinal mucosa is invaded and damaged by a protozoa of the subclass Coccidia. The economic effects of coccidiosis can be especially severe in the poultry industry where intensive housing of birds favors the spread of the disease. Infection by coccidial protozoa is, for the most part, species specific. Numerous species, however, can infect a single host. For example, there are seven species of coccidial protozoa which infect chickens, six of which are considered to be moderately to severely pathogenic.

The life cycle of the coccidial parasite is complex. For example, protozoa of the genera Eimeria, Isospora, Cystoisospora, or Cryptosporidium typically only require a single host to complete their life cycle, although Cystoisospora may utilize an intermediate host. Under natural conditions, the life cycle begins with the ingestion of sporulated oocysts from the environment. When sporulated oocysts are ingested by a susceptible animal, the wall of the sporulated oocyst is broken in order to release the sporocysts inside. In poultry, the release of the sporocyst is the result of mechanical disruption of the sporulated oocyst in the gizzard. Within the sporocysts, are the sporozoites which are the infective stage of the organism. In poultry, the breakdown of the sporocyst coat and release of the sporozoites is accomplished biochemically through the action of chymotrypsin and bile salts in the small intestine. Once released, the sporozoites invade the intestinal mucosa or epithelial cells in other locations. The site of infection is characteristic of the species involved. For example, in the genus Eimeria, E. tenella is localized in the ceca; E. necatrix is found in the anterior and middle portions of the small intestine; E. acervulina and E. praecox occur in the upper half of the small intestine; E. brunetti occurs in the lower small intestine, rectum, ceca, and cloaca; E. mitis is found in the lower small intestine, while E. maxima can be found in any of these physiological locations.

Once inside the host animals' cells, sporozoites develop into multinucleate meronts, also called schizonts. Each nucleus of the meront develops into an infective body called a merozoite which enters new cells and repeats the process. After a variable number of asexual generations, merozoites develop into either microgametocytes or macrogametes. Microgametocytes develop into many microgametes which, in turn, fertilize the macrogametes. A resistant coat then forms around the resulting zygotes. The encysted zygotes are called oocysts and are shed unsporulated in the feces. Infected birds may shed oocysts in the feces for days or weeks. Under proper conditions of temperature and moisture, the oocysts become infective through the process of sporulation. Susceptible birds then ingest the sporulated oocysts through normal pecking activities or ground/litter foraging and the cycle repeats itself. Ingestion of viable, sporulated oocysts is the only natural means of infection.

Infection with coccidial protozoa results in immunity so that the incidence of the disease decreases over time as members of the flock become immune. This self-limiting nature of coccidial infections is widely known in chickens and other poultry. The immunity conferred, however, is species specific such that introduction of another species of coccidial protozoa will result in a new disease outbreak.

The oocyst wall of coccidial protozoa provides a highly effective barrier for oocyst survival. Oocysts may survive for many weeks outside the host. In the laboratory, intact oocysts are resistant to extremes in pH, detergents, proteolytic, glycolytic, and lipolytic enzymes, mechanical disruption, and chemicals such as sodium hypochlorite and dichromate.

Two methods are currently used to control coccidiosis in poultry. The first involves control by chemotherapy. Numerous drugs are available for the control of coccidiosis in poultry. Because of the number of species which cause the disease, very few drugs are efficacious against all species, although a single drug may be efficacious against several species. In modern broiler chicken production, for example, administration of drugs to control coccidiosis is routine. The expense for preventative medication against coccidiosis represents a significant cost of production.

Two programs of drug administration are commonly used in the domestic poultry industry. The simplest is the continuous use of a single drug from day one following hatching until slaughter. The second program is to use shuttle or dual drug program which involves the use of two different drugs, one administered in the “starter” ration and a second drug administered in the “grower” ration. This second method is often preferred as a method to minimize development of drug resistant strains of Coccidia. Using either method, drugs used are typically rotated two to three times per year in order to minimize the development of resistant strains.

The development of drug resistance by Coccidia is a serious limitation on the effectiveness of chemotherapy to control the disease. Surveys in the United States, South America and Europe have revealed widespread drug resistance in Coccidia. Since drug resistance is a genetic phenomenon, once established, drug resistance can remain in the population for many years until reduced by natural selection pressure and genetic drift.

The use of drugs in animals used for food production is also coming under increasing scrutiny by the public. Consumers are increasingly concerned with the possibility of drug residues in food. This creates pressure in the poultry industry to reduce the use of drugs to control coccidiosis.

Vaccination of birds against coccidiosis is an alternative to chemotherapy. An advantage of vaccination is that it can greatly reduce or eliminate the need to administer anti-coccidial drugs, thus reducing drug costs to poultry producers, preventing the development of drug-resistant strains, and lessening consumer concerns about drug residues.

Numerous methods have been developed to immunize poultry against coccidial protozoa. The successful methods have all been based on the administration of live protozoa, either fully virulent strains or attenuated strains. The most common route of administration is oral, although other routes have been used. Edgar, U.S. Pat. No. 3,147,186, teaches vaccination of chickens by oral administration either directly into the mouth or via the feed or water of viable E. tenella sporulated oocysts. Davis et al., U.S. Pat. No. 4,544,548, teaches a method of vaccination by continuous administration of low numbers of sporulated oocysts, with or without simultaneous administration of anti-coccidial drugs.

Oral administration of attenuated strains of sporocysts has also been utilized to confer immunity against coccidiosis. Shirley, U.S. Pat. No. 4,438,097; McDonald, U.S. Pat. No. 5,055,292; and Schmatz et al., PCT publication No. WO 94/16725. An alternative to attenuation is disclosed in Jenkins et al., Avian Dis., 37(1):74-82 (1993), which teaches the oral administration of sporozoites that have been treated with gamma radiation to prevent merogonic development.

Parenteral routes of vaccination have included subcutaneous or intraperitoneal injection of excysted sporozoites, Bhogal, U.S. Pat. No. 4,808,404; Bhogal et al., U.S. Pat. No. 5,068,104, and intra ovo injection of either oocysts or sporocysts, Evans et al., PCT publication No. WO 96/40233; Watkins et al., Poul. Sci., 74(10):1597-602 (1995). Sharma, J. Parasitol., 50(4):509-517 (1964), reported unsuccessful immunization trials involving intravenous, intraperitoneal, intramuscular, or subcutaneous injection of either viable oocysts or a mixture of oocysts, sporocysts and sporozoites. Thaxton, U.S. Pat. No. 5,311,841, teaches a method of vaccination against Coccidia by administration of oocysts or sporozoites to newly hatched chicks by yolk sac injection.

Regardless of the route of administration, procedures for the production of coccidiosis vaccines are quite similar. Briefly, coccidial protozoa are produced by infecting host animals with a single species of coccidial protozoa. These “seed stocks” are often clonal in nature, that is, derived from a single organism in order to insure the presence of only the species of interest. Seed stocks may be wild type, that is, isolated from the field, or they may be precocious or attenuated strains. The protozoa are then allowed to undergo replication in the host, after which, protozoa are collected from the animals, usually from the excreta. The use of attenuated strains typically results in fewer shed oocysts from the host animal. The protozoa are then separated from the excreta by well known techniques such as salt flotation and centrifugation. At the time of collection, the protozoa are at the non-infective oocyst stage of the life cycle. In order to become infective, and therefore useful for vaccines, the oocysts must be induced to undergo sporulation. In members of the genus Eimeria, sporulation typically involves incubating the oocysts in a 1% to 4% aqueous solution of potassium dichromate at 19° C. to 37° C. with constant aeration. Data on oxygen consumption are conflicting, with Smith and Herrick (J. Parasitol. 30:295-302, 1944) reporting increased oxygen consumption for E. tenella during sporulation and Wilson and Fairbairn (J. Protozool. 8:410-416, 1961) reporting no change in oxygen consumption for E. acervulina. Sporulation is usually complete within 12 to 24 hours depending on the temperature used. Monitoring of the sporulation process is accomplished by microscopic examination of the protozoa. Current vaccines available for the prevention of coccidiosis typically contain a 2.5% weight to volume solution and contain approximately 1,600 oocyts per dose (400 sporulated oocysts representing each of four different species).

One problem associated with the administration and storage of vaccines comprising oocysts, is the tendency of the oocysts to aggregate (i.e. clump or stick) to each other, or to aggregate at an interface. For example, oocysts may aggregate at the container cap or stopper, at the vaccine/air interface or vaccine/container interface, or stick to each other, forming clumps. Such aggregation can result in non-uniform administration of the vaccine, which in turn may lead to over or under dosing of the vaccinated animal.

It is thus desirable to prevent or eliminate oocyst aggregation, and thus provide uniform administration of the vaccine to the host animal.

### SUMMARY OF THE INVENTION

The present invention thus provides a composition comprising viable sporulated oocysts of at least one species of protozoa known to cause coccidiosis, a pharmaceutically acceptable carrier, diluent, or excipient, and at least one surfactant capable of preventing or reducing the aggregation of sporulated oocysts. Preferably the composition is sterile.

Also provided is a preparation for the prevention and treatment of coccidiosis comprising a pharmaceutically acceptable carrier, diluent, or excipient; live sporulated oocysts of at least one species of coccidial protozoa, and an amino acid, wherein the sporulated oocysts are sanitized.

Other aspects will be apparent in light of the following description and claims.

### BRIEF DESCRIPTION OF THE DRAWINGS

FIG. 1 shows a graph of percent saturation of dissolved oxygen (A) and pH (B) versus time of the sporulation medium during a successful sporulation when percent saturation of dissolved oxygen is not controlled.

FIG. 2 shows a graph of percent saturation of dissolved oxygen (A) and pH (B) versus time of the sporulation medium during a successful sporulation in which the rise in pH was preceded by a drop in pH when percent saturation of dissolved oxygen and pH is not controlled.

FIG. 3 shows the percent viable oocysts recovered (PVOR) versus storage time for oocytes stored under the conditions indicated. All storage medium contained P. acnes and oocysts were sterilized by 5% NaOCl.

FIG. 4 shows the percent viable oocysts recovered (PVOR) versus storage time for oocytes stored under the conditions indicated. All storage medium contained P. acnes and oocysts were sterilized by 2% NaOCl.

FIG. 5 shows a flow diagram of the process used to produce oral coccidiosis vaccine. The flow chart is divided into four suites: (1) Challenge Suite; (2) Purification Suite; (3) Sporulation Suite; and (4) Storage Suite. Steps 1 through 34 are described for illustrative purposes as follows:

1. Feed given to the birds

2. Water given to the birds

3A. Manure is harvested after birds begin shedding oocysts

3B. Manure is discarded prior to when birds shed oocysts

4. Water is added to a slurry tank containing the manure

5. The slurry is sieved

6. Solid waste from sieving is discarded

7. Filtrate collection transferred to and then separated by centrifugal-based separation

8. Liquid waste is discarded while moist solid is retained

9. Moist solid is transferred to mix tank

10. High fructose corn syrup is added to tank

11. Water is added (if needed) to adjust specific gravity

12. Suspension is transferred for separation

13. Solid wastes are discarded

14. Liquid phase is transferred to mix tank

15. Water is added to mix tank

16. Diluted suspension is transferred to and then centrifuged

17. Liquid waste is discarded

18. Solid transferred to blend vessel

19. Water is added to blend vessel

20. Diluted suspension is deposited in holding container then later transferred to sporulation vessel

21. An oxidizing agent is added to the sporulation medium

22. Sporulated oocysts transferred from sporulation vessel to separation device

23. Water is added to sporulated oocysts suspension

24. Waste water from separation is removed and discarded

25. Sporulated oocysts, now separated from sporulation medium, are transferred to sterile filtration unit

26. Addition of disinfecting agent

27. Addition of sterile water

28. Waste water and disinfecting agent are discarded

29. Transfer to holding vessel

30. Addition of buffer

31. Addition of bactericide

32. Transfer to blending vessel where buffer and post-challenge performance improvement composition are blended

33. Vialing

34. To kit or to market

### DETAILED DESCRIPTION OF THE INVENTION

It has been discovered that when a vaccine composition comprising sporulated oocysts is agitated, and the oocysts repeatedly contacted with air or other gas above the aqueous phase, some oocysts tend to aggregate at the composition/air interface (i.e. at the composition surface), at a composition/surface interface (e.g. composition/container interface), or stick to each other, forming clumps. At times the oocysts will also aggregate at the container surface above the liquid level, or on the container cap or stopper. Severe aggregation of oocysts can affect the vaccine dosage, and can lead to over or under dosing of the vaccinated animal. The present invention provides vaccine compositions and methods that address these problems.

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, databases and other references cited in this application are herein incorporated by reference in their entirety as if each individual publication, patent, patent application, database or other reference were specifically and individually indicated to be incorporated by reference.

Definitions

The terms “aggregation,” “accumulation,” “clumping,” or “sticking,” used interchangeably herein, refer to the amassing or grouping together of oocysts, sporocysts, or sporozoites within a composition. Aggregation may occur at an interface, or may occur on a cap or stopper of the vaccine or composition container. Aggregation also refers to the clumping or sticking together of oocysts, sporocysts, or sporozoites in a composition.

The term “bacterial contamination” or “bacterial contaminants” as used herein means all extraneous bacterial contaminants, whether live, virulent, and infectious, or lifeless or non-virulent, including cellular debris derived from such extraneous bacterial contaminants. “Substantially free of bacterial contamination” or “substantially free from bacterial contaminants” means below the level that can create a pyrogenic reaction when a vaccine of the invention is administered to poultry. Methods for determining whether a pyrogenic reaction occurs are well known in the art. In any event, a composition is substantially free of bacterial contamination when no bacterial contaminants, as defined herein, are visible upon microscopic examination of the composition, wherein the detection limit is at least about 1,100 bacteria per milliliter.

The term “cap” or “stopper,” used interchangeably herein, means a protective covering or seal that closes off the end of a container.

The term “interface” means any point at which the liquid medium of a vaccine or composition, as described herein, comes into contact with one or more phase, such as another liquid, a surface, or a gas (e.g. air). Examples of an interface include an interface between a vaccine or composition and air, and an interface between a vaccine or composition and a surface (e.g. the surface of the container in which the vaccine is held, or the container cap or stopper).

The term “minimum effective surfactant concentration” means the minimum concentration of surfactant that will be effective for reducing or preventing the aggregation of oocysts, sporocysts, or sporozoites.

The term “oocyst” means the life-cycle stage of a protozoan having a tough outer coat. The term “oocyst” is meant to includes sporulated as well as unsporulated oocysts.

The term “sterile” or “sanitary” means that there are no detectable amounts of live, viable, bacteria, viruses, or fungi in the composition.

Vaccines

As previously indicated, when administering a vaccine composition comprising oocysts, sporocysts, or sporozoites to an animal, it is important that the oocysts, sporocysts, or sporozoites be uniformly distributed throughout the vaccine composition. Aggregation of oocysts, sporocysts, or sporozoites can occur at an interface, such as at the vaccine-air interface, the vaccine-container interface, on a wetted container cap or stopper, or the oocysts, sporocysts, or sporozoites can clump to each other. If the oocysts, sporocysts, or sporozoites have aggregated, there is a lack of uniform distribution of the oocysts, sporocysts, or sporozoites which may lead to over or under dosing of the vaccinated animal. It is thus desirable to prevent oocyst, sporocyst, and sporozoite aggregation in vaccines. Likewise, oocyst, sporocyst, or sporozoite aggregation may occur when the oocysts, sporocysts, or sporozoites in a vaccine composition are exposed to air, such as when the vaccine is shaken. In such an instance, it is desirable to reduce, or eliminate entirely, any oocyst, sporocyst, or sporozoite aggregation that has occurred. It has been discovered that maldistribution of oocysts, sporocysts, and/or sporozoites can be prevented or reduced by the use of the surfactants described herein; and that formulations comprising oocysts, sporocysts, and/or sporozoites, and further containing such surfactants, can be effectively used in treating or preventing coccidiosis.

A variety of vaccine compositions comprising oocysts, sporocysts, or sporozoites are known in the art, some of which are described above, and may be used in combination with a surfactant described herein. Particularly preferred are coccidiosis vaccines. Such vaccines may comprise oocysts, such as sporulated oocysts, sporocysts, sporozoites, or any combination thereof. Particularly preferable are the compositions described in U.S. application Ser. No. 10/728,194, herein incorporated by reference in its entirety.

Thus, in one aspect, the present invention provides a composition comprising viable oocysts, sporocysts, sporozoites, or any combination thereof, of at least one species of protozoa known to cause coccidiosis. The composition preferably further comprises a pharmaceutically acceptable carrier, diluent, or excipient, and at least one surfactant capable of preventing or reducing oocyst aggregation. Preferably, the oocysts are sporulated oocysts.

In addition to providing vaccine compositions, the present invention further provides methods for the prevention and treatment of coccidiosis, such as in members of the class Aves. The methods involve administering or offering to said member a composition of the present invention. As previously indicated, the compositions preferably comprise a pharmaceutically acceptable carrier, diluent, or excipient, and at least one surfactant, as described herein, that is capable of preventing or reducing the aggregation of oocysts, sporocysts, sporozoites, or any combination thereof. Preferably, the composition comprises viable, oocysts, sporocysts, sporozoites, or any combination thereof, of at least one species of protozoa known to cause coccidiosis.

The compositions and methods of the present invention are described in further detail below. It is noted that although the compositions are described herein in terms of compositions comprising oocysts, and in particular sporulated oocysts, the present invention is also applicable to vaccine compositions comprising sporocysts, sporozoites, or any combination of oocysts, sporocysts, and sporozoites, as well.

Methods of producing vaccines comprising sporocysts or sporozoites are known in the art. For example, U.S. application Ser. No. 09/708,918, herein incorporated by reference in its entirety, describes a method for treating sporulated oocysts to disrupt the oocyst wall to release sporocysts. Other methods of production may also be used.

Methods for producing vaccines comprising oocysts are also known in the art. Preferably, the vaccine composition of the present invention is produced by the method described in U.S. Application Pub. No. US2002/0160022 Al, incorporated herein by reference in its entirety. This method is described below.

In one aspect, the vaccine compositions of the present invention may comprise at least one species of protozoa known to cause coccidiosis. Any source of oocysts of a single species can be used herein. Preferably, the oocysts are from a coccidial species of the order Eucoccidiida. More preferably, the coccidial oocysts are of the genus Eimeria. Even more preferably the species of coccidial oocysts are selected from the group consisting of Eimeria acervulina, Eimeria maxima, Eimeria mitis, Eimeria tenella, Eimeria necatrix, Eimeria brunetti, or Eimeria praecox. The vaccine composition of the present invention may also consist of a plurality of species of protozoa. In this instance, the plurality of species preferably comprises the combination of E. tenella, E. maxima, and E. acervulina.

Protozoan strains used in coccidiosis vaccines include precocious strains, attenuated strains, and wild type strains, such as field isolates. The Eimeria strains preferably used in the compositions of the present invention can be obtained from a variety of sources. For example, oocysts can be obtained by the inoculation of host animals with coccidial protozoa of a single species or with a mixture of species. The coccidial protozoa used can be clonal in nature, that is derived from a single progenitor, or polyclonal. The oocysts used herein may be wild type oocysts. Preferably, the oocysts of the present composition are derived from wild type oocysts. Inoculation can be by any means which will allow for the replication of the protozoa in the host animal. The most common route of inoculation is per os, but other suitable routes may be used. If administered per os, the protozoa are preferably at the sporulated oocyst stage. Administration can be by gavage or through the feed and/or water. Inoculation can also be accomplished by exposing host animals to environments contaminated with coccidial protozoa. Alternatively, oocysts can be obtained from animals with naturally occurring infections.

Methods of Isolation, Concentration, and Purification

In general, a number of different methods of preparing oocysts for sporulation are known in the art. Any one or combination of such methods may be used prior to sporulation. However, a preferred method is set out below. A number of well known processes are set forth to assist one skilled in the art to practice the invention in its different embodiments.

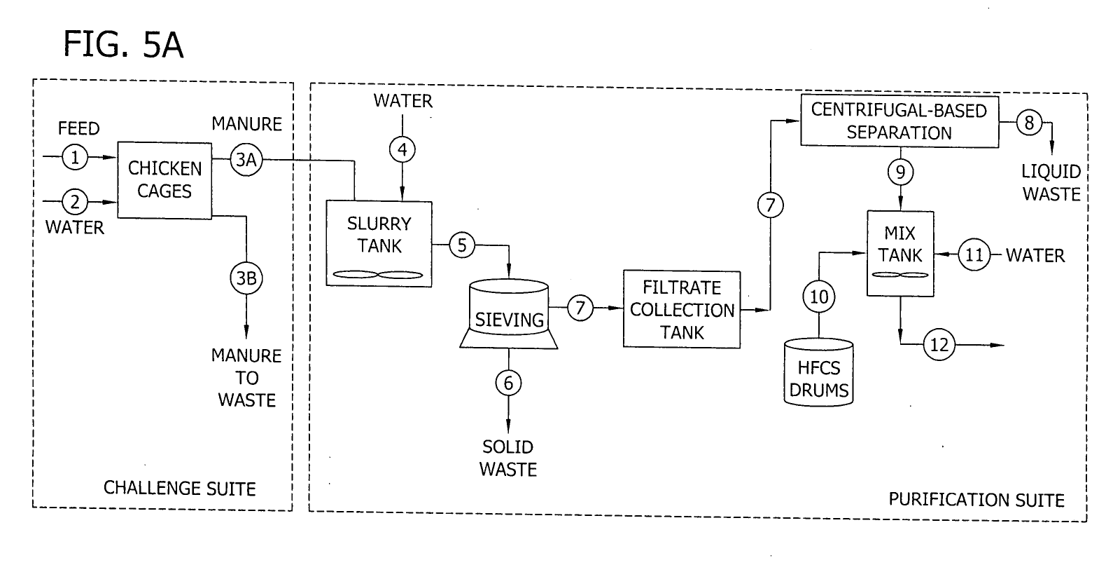

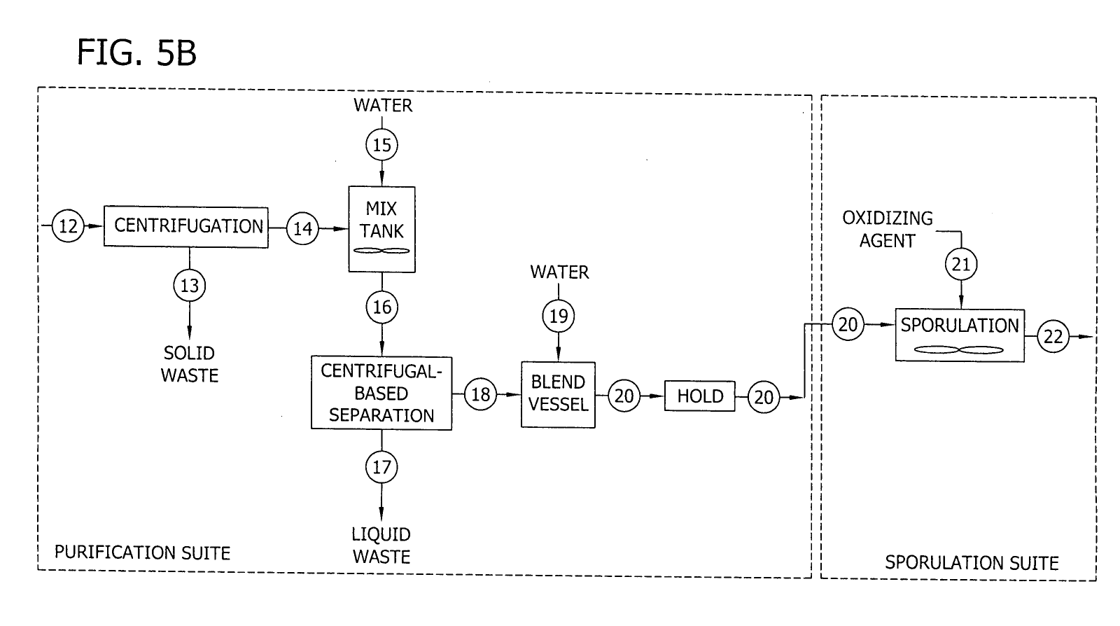

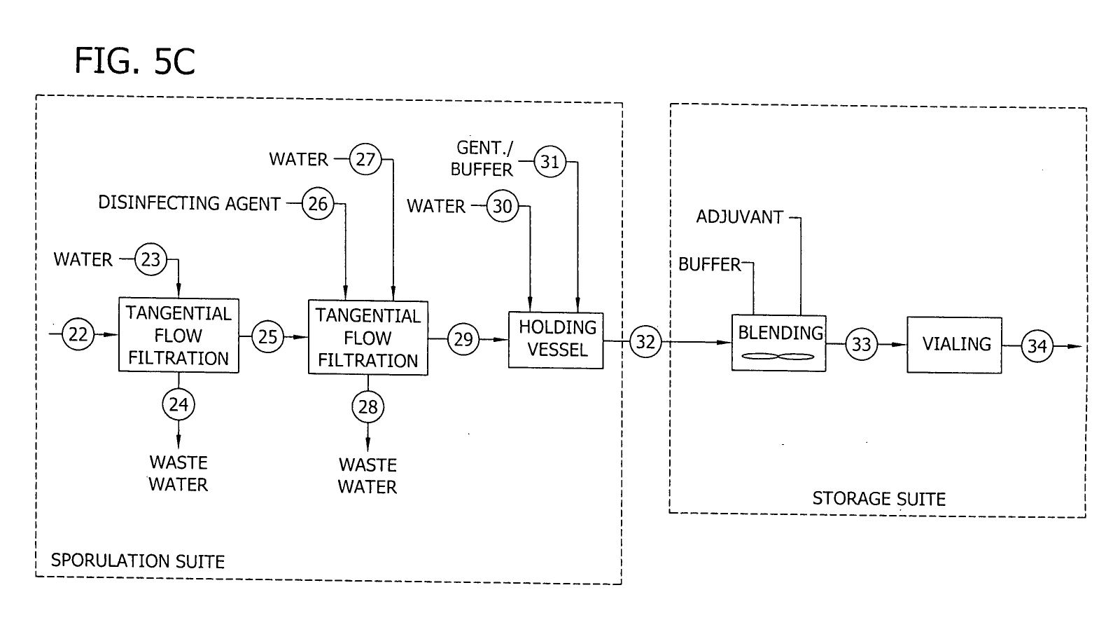

To begin, once host animals begin shedding the organism, the protozoa can be collected. Most commonly, protozoa are collected from the feces, but they can also be collected from intestinal contents and/or scrapings as well as contaminated bedding (see FIG. 5A, “Challenge Suite”). Once collected, the oocysts are preferably isolated from the extraneous fecal material, as decreasing the fecal content in an oocyst suspension increases the number of oocysts that will sporulate (Smith and Ruff, Poultry Sci. 54:2083, 1975). As will be described below, a preferred method for isolating oocysts is by sieving (see FIG. 5A, step 5). However, several methods for isolating protozoa are known in the art and may be used in practicing the present invention. These isolation methods are summarized herein followed by a description of the preferred method. Several methods described herein process the collected manure to a point wherein sporulation may then begin. Others require further processing, such as further isolation or cleansing. This further processing may be accomplished by utilizing the methods and techniques described herein or a combination thereof.

A review of several methods for the isolation of oocysts can be found in Ryler et al.(Parasitology 73:311-326, 1976). In one method, described in U.S. Pat. No. 3,147,186, oocysts are only crudely isolated following the addition of the oxidizing agent potassium dichromate. In this method, the moist droppings of host animals are directly mixed with an aqueous solution containing between one and four percent potassium chromate, preferably 2.5% or, less preferably, water, so that a suspension of thin consistency is obtained. The method indicates that a concentration of at least about one to four percent potassium chromate solution is necessary to obtain adequate oocyst sporulation. Larger insoluble debris, such as feathers and partially digested or undigested feed, is removed. Removal can be done conventionally by filtering the suspension through a mesh screen. The suspension is then allowed to stand for about five minutes to allow heavier coarser particles of debris that passed through the screen to settle to the bottom of the holding container. The supernatant liquid containing the oocysts is then removed. The sporulated oocysts are viable for up to about 18 hours.

Another method for separating oocysts from droppings comprises flotation using solutions of sufficient specific gravity, typically having a specific gravity of about 1.2, so that oocysts float to the top of the suspension. Generally these solutions are made up of water to which a sugar (e.g. sucrose), ZnSO₄, or NaCl has been added to increase the specific gravity to the desired value. Useful solutions include solutions comprising 58% (w/v) sucrose, 37% (w/v) ZnSO_{4·}7H₂O and saturated NaCl solutions, which all have a specific gravity from about 1.09 to about 1.2. Other solutions which have a comparable specific gravity and are not harmful to the oocysts can also be used.

In the flotation method of isolation, a preliminary step of filtering diluted collected manure through, e.g., gauze, a sieve or cheesecloth to remove large particles of undesired fecal matter may be included. After mixing harvested oocysts with the flotation solution, the oocyst slurry may be centrifuged and the oocyst removed from the surface layer of the supernatant. The centrifugation step may be repeated several times to further purify the oocysts by resuspending the captured supernatant in a flotation medium having a specific gravity similar to that used in previous centrifugation steps and centrifuged again. This step may be repeated until the desired level of purity is reached.

Another method for isolation of oocysts available in the art comprises gradient centrifugation. The gradient used can be discontinuous or continuous. An example of a typical gradient for coccidial oocysts is 0-50% sucrose. In this method the material containing the oocysts is placed on top of the gradient and the oocyst containing material is then centrifuged along with the gradient. Following centrifugation, the layer containing the oocysts is recovered. The process may be repeated in order to increase the purity of the resulting oocyst preparation. As with flotation, this method is preferably preceded by filtration of the collected manure.

Additional methods of oocyst isolation include, the use of glass bead columns (Ryler et al., Parasitology, 73:311-326, 1976) and the bicarbonate ether-method (Smith and Ruff, Poultry Sci. 54:2081-2086, 1975). In the glass column method, the aqueous suspension of fecal matter is added to a mixture of glass beads and a detergent, for example 5% Tween 80. The mixture is then applied to a column of glass beads and the oocysts are allowed to flow through while much of the undesired fecal matter is retained in the column. The effluent may then be concentrated by centrifugation.

In the bicarbonate ether method, the feces from infected chickens is strained, through cheese cloth for example, and the liquid fraction is captured while the solid fraction is discarded. The liquid fraction is then concentrated by centrifugation. The solid fraction is recovered and the supernatant is discarded. The recovered solid fraction is then resuspended in a solution of 1% sodium bicarbonate. To the resuspended solid fraction, now in suspension, is then added ether in a volume approximately equal to the volume of 1% solution of sodium bicarbonate. The mixture is then centrifuged. The debris plug and supernatant is discarded while the sediment is washed by resuspension in water. This suspension is then centrifuged and the supernatant discarded. The sediment is then recovered for use. (Smith and Ruff, Poultry Sci. 54:2081-2086, 1975).

The various methods for isolating, concentrating, and purifying oocysts described above may be used in combination with one another or in combination with the preferred method described below. Regardless of the methods used, the greater the isolation, concentration, and purification the greater percent sporulation during the sporulation suite (see FIG. 5, “Sporulation Suite”). Therefore, it has been discovered that the following methods of isolation, concentration, purification, sporulation, sterilization, and storage provide a novel and improved method for the production of sporulated oocysts.

The preferred methods for isolation, concentration, flotation, sporulation, monitoring, separation, and sterilization, are now described.

Oocyst Isolation

The initial isolation of oocysts from the gross fecal matter is desired to remove large debris and fecal matter from a manure slurry. Thus, the step begins with a manure slurry highly contaminated with gross particles and results in an aqueous manure slurry substantially free of gross particles. Although isolation may be achieved by one of the above methods known in the art, isolation in the present invention may be accomplished by filtering. Preferably, the filtration is by sieving.

The initial isolation may be achieved by collecting manure from host animals, mixing the manure with domestic water, and then sieving. For example, the process may begin with collected manure, e.g., a batch of several hundred pounds, made into a aqueous slurry and processed to concentrate oocysts as a suspension in a relatively small volume of aqueous medium, e.g., a several hundred pound batch of manure may ultimately yield about two liters of oocysts in an aqueous suspension (see FIG. 5A, “Challenge Suite”, steps 1-3B). Sieving is preferably by means of shaker screens, such as multiple tier shaker screens.

The manure from the chickens may be placed into a mixing vessel either by hand, e.g. using a shovel, or by using a mechanical dumper. Domestic water may be added at a minimum ratio of about 1 gallon per each six birds' manure (see FIG. 5A, steps 3A and 4). Alternatively, the collected manure may be mixed with domestic water at a ratio from about 2 to about 6 pounds of collected manure per gallon of water. As manure quantity is approximate and dilution is realized by using an approximation of the manure quantity, the dilution range is therefore also approximate. The slurry may then mixed until homogeneous. Feathers and other large, floating debris may be skimmed off the surface with an appropriate tool, e.g., a wire screen.

Typically, a sample is taken from the homogenous slurry prior to the screening/isolation process to assess oocyst count. Such count is done, for example, by microscopic visual examination. The homogenous slurry may then be pumped onto a two-tier vibratory shaker screen. The top screen can be from about 150-mesh to about 350-mesh while the bottom screen can be from about 25-mesh to about 75-mesh. Preferably, the top deck is equipped with a 50-mesh screen, the “top screen,” while the lower deck has a 250-mesh screen, the “bottom screen.” Larger unwanted fecal solids are separated at the top deck 50-mesh screen while smaller unwanted fecal solids are separated from the slurry at the lower deck, 250 mesh screen. A preferred flow rate onto the top deck screen is approximately 1 liter per minute per 12.1 m². The optimal flow rate of the pumping can vary with the solids content and the condition of the screen. Larger or smaller screens may be used depending on the scale of the operation.

The oocysts are contained in the liquid fraction of the screening/isolation process. If the solid material coming off either the top or the bottom screen is too wet, recovery may be unacceptably low as isolation of the oocysts from the homogenous slurry may not be occurring. On the other hand, if too much water is removed, the solids may stick to the screen and not clear themselves. Eventually, depending on solid matter content and flow rates, so much material can accumulate that it may be necessary to remove it. The screens can be lubricated with water, allowing the screens to clear themselves. The liquid fraction may be collected and sent on for concentration while the solid fraction is discarded (see FIG. 5A, step 6).

Preferably, the solids coming off both of the screens are checked for oocysts and then discarded. If less than 5% of the oocysts loaded onto the screens are found in the solids, then the solids are typically discarded. If more than 5% of the oocysts loaded onto the screens are found in the solids, the solids are typically resuspended in an aqueous slurry and recycled through the sieves. The liquid that passes through both screens, the filtrate, is the fraction that contains the oocysts. This filtrate may be collected into a receiving vessel (see FIG. 5A, step 7) and then sent to a centrifuge, preferably a bottle centrifuge or, alternatively, a decanter centrifuge.

In this non-limiting example, the sieving method can be carried out in temperatures ranging from a low temperature that substantially avoids freezing to a high temperature that substantially avoids damage to the oocysts, preferably at room temperature. Lower temperatures, about 40° C., are preferred when sieving procedures take over more than three hours, to protect the viability of the oocysts. In addition, the sieving process can be carried out at any rate throughput allows as long as the screens do not accumulate excessive solid matter and at a rate rapid enough to prevent the manure from drying. As with other steps of the invention, equipment is preferably cleaned prior to use.

Concentrating the Filtrate

The liquid oocyst-containing fraction recovered, from any one of the preceding methods used to initially isolate oocysts, may then be concentrated. Isolated oocysts are concentrated to increase sporulation rates and output. Concentration can be realized by various means, including substantial separation from the aqueous slurry, centrifugal-based separation or centrifugal-based separation followed by filtration. Concentrating the oocysts is preferably accomplished by utilizing one or more of such techniques in combination with others. As used herein, centrifugal-based separation includes processing in either a mechanical rotated centrifugation or a static hydrocyclone. Centrifugation may be by decanter centrifugation. Other methods are also known in the art and disclosed herein. Centrifuge scale and capacity varies by batch size. For larger batch size, the use of a decanter centrifugation or use of a hydrocyclone would be preferred. For smaller batch size, bottle centrifugation is preferred.

In one method, a combination of first sieving the collected manure, as described in the preferred sieving process above, is followed by continuous centrifugation and then filtration. In this method preliminary purification is achieved by sieving a homogenous slurry of collected manure through sieves having progressively smaller openings. Further purification can be achieved by continuous centrifugation of the liquid fraction captured from the sieving process using a suspension wherein the suspension has a specific gravity preferably between about 1.01 to about 1.08 g/l. As a further purification step, the solid material recovered from the centrifugation, which contains the oocysts, is preferably re-slurried and filtered using a membrane of a pore size that retains the oocysts, but allows the passage of smaller material, including bacteria. This process eliminates the flotation step, as described below.

In an example of a further alternative procedure, the material passing through the sieves can be further purified by being collected and pumped into a continuous flow centrifuge maintained at about 40-50° F., discarding the centrate, and collecting the solid material containing the oocysts (such as seen in FIG. 5A, step 8).

Preferably, the filtrate recovered from the multi-screen screening/isolation process described above is then concentrated using a centrifuge. The filtrate recovered from the multi-screen screening process described above may then be concentrated using a bottle centrifuge. Preferably, the filtrate recovered from the isolation/screening process is then concentrated using a bottle centrifuge wherein the filtrate is poured into centrifuge bottles and centrifuged at 1200× g for about ten minutes. The solid fraction formed contains the oocysts. The supernatant may then be poured off (see FIG. 5A, step 8) and, if the solid fraction volume allows, more filtrate can be poured on top of the solid fraction. At some point, the solid fraction will need to be removed as more oocysts collect. The oocysts may be loosened or removed with a spoon or spatula. Residual material that is still in contact with the centrifuge bottles may be rinsed out using a minimal amount of domestic water. The oocysts may then require suspension in sufficient water to bring the solids content to less than about 70% of the total volume of the suspension, preferably less than about 60% of the total volume, and most preferably less than about 50% of the total volume, and should bring the suspension to substantial homogeneity. During this suspension process, mixing of the aqueous suspension is preferably sufficient to keep the solids suspended, but the mixing should not create foaming. This suspension may then be further processed by the flotation step, described herein below.

In an alternative preferred example, the filtrate from the multi-screen screening process described above, can be pumped into a decanter solid bowl continuous centrifuge at a rate of approximately 3 to 4 liters/min. The decanter is preferably set with a bowl speed of at least about 4000 RPM and a conveyor speed of at least about 2500 RPM and no more than about 3600 RPM. A receiving vessel is placed to catch the solids as they are expelled from the solid discharge of the decanter centrifuge. Under these conditions, solids are typically discharged as a runny paste. The liquid coming out of the liquid discharge of the decanter centrifuge is preferably checked for oocysts and discarded if the liquid contains less than about 2% of oocysts initially loaded into the decanter centrifuge. If the liquid waste contains greater than or about 2% of oocysts initially loaded into the decanter centrifuge, the liquid is typically re-mixed with the solids and run again until the liquid waste contains less than 2% of oocysts initially loaded into the decanter centrifuge.

Preferably, once all the filtrate is pumped into the decanter centrifuge, the centrifuge is allowed to run for a period of time sufficient to move the residual solids out of the decanter. This period of time is preferably more than about two but less than about five minutes. Use of various sized centrifuges can vary the period of time and may be adjusted by one skilled in the art. Preferably, the speed of the bowl is then lowered to about 1000 RPM and the speed of the conveyor lowered to about 500 RPM. The length of time and the bowl speed also varies according to batch size and can be properly adjusted by one skilled in the art. Domestic water may be sprayed into the access port to wash solids off the inner surfaces of the body of the decanter. The solids can then be moved to an appropriate volume centrifuge bottle for the flotation step, described below. Typically, it is important to clean the equipment after each run, and may be accomplished by the use domestic water sprayed from a hose in order to obtain greater yield.

In another example, a hydrocyclone can be used to concentrate the filtrate obtained from sieving. It has been discovered that a hydrocyclone, traditionally used in the petrochemical and environmental science fields is useful for concentrating oocysts. Hydrocyclones use the principle of centrifugal separation to remove or classify solid particles from a fluid, based on size, shape, and density. The use of a hydrocyclone, not known to be used for living organisms, was previously believed to fatally damage the oocysts due to intense sheer forces. The instant invention provides a method of utilizing a hydrocyclone to concentrate oocysts. The hydrocylone used is preferably a Dorr-Oliver DOXIE Type 5 Hydrocyclone (available from GL&V/Dorr-Oliver, Millford, Conn.).

Preferably, when a hydrocyclone is used, a reservoir containing the filtrate obtained from sieving is connected to a pump. The pump preferably delivers the filtrate to the hydrocyclone at a pressure of between about 120 psi and about 130 psi and at a feed rate from about 1 to about 3 gallons per minute, preferably about 2 gallons per minute. A preferred hydrocyclone has one inlet and two outlets. Each outlet is equipped with a needle valve to regulate the flow through each orifice. By regulating the flow between the upper and lower outlets, it is possible to remove a significant amount of liquid through the upper outlet while retaining most of the denser materials, including the oocysts, in a concentrated suspension through the lower outlet. Preferably, there is a 2 to 1 ratio between the flow of material collected from upper outlet and lower outlet. Such 2 to 1 ration typically produces an optimal recovery of oocysts. The recovered concentrated material, that is, the material collected from the lower outlet, may be recycled through the hydrocyclone for greater concentration if further volume reduction is desired. The suspension collected from the upper outlet may be discarded. For large volumes of filtrate, it may be advantageous to operate hydrocyclones in parallel or utilize larger scale equipment to increase throughput.

Floating the Oocvsts

To further isolate the oocysts collected from the concentration methods described above from unwanted solids, such as fecal matter, grit, etc., the oocysts may be floated to the top of a solution using density variations. Alternatively, the oocysts may be added to a sucrose solution and centrifuged. The oocysts may also be added to a sucrose solution and then filtered. Preferably, the oocysts are floated to the top of a solution comprising domestic water and high fructose corn syrup and having sufficient density to allow the oocysts to float to the top of the suspension while the heavier unwanted solids migrate to the bottom of a holding vessel or vessels. Preferably, the oocysts are isolated from the dense solutions using centrifugation. The oocysts may then be recovered from the liquid phase in this step of the invention.

The solid material containing the oocysts recovered from centrifugation is transferred to a mix tank and to a concentrated sucrose or high fructose corn syrup (HFCS) of volume equal to that of the oocysts is added. Preferably, a water of a volume equal to that of the oocyst/HFCS solution is added for a total final volume of about four times the volume of the initial solids (FIG. 5A, steps 9-11). The final mixture is then preferably pumped into a continuous centrifuge at a rate to allow the oocysts to remain in the centrate, and solids are discarded (FIG. 5B, steps 12-13). If desired, further concentration of the oocysts and dilution of and substantial removal of the residual sugar solution may be accomplished by addition of domestic water and continuous flow centrifugation at a feed rate which allows separation of the phase containing the oocysts from the sugar solution phase. Alternatively, the oocysts/HFCS solution can be centrifuged in a bottle centrifuge. In this case, the supernatant is discarded and the oocysts in the resulting solid fraction are resuspended in water. The residual sugar can also be removed by filtration using filters with a pore size which excludes the oocysts. When filtration is used, tangential flow is preferred. Tangential flow filtration (“TFF”) is characterized in that an influent stream is separated into two effluent streams, known as permeate and retentate. The permeate is that fraction which has passed through the “semi-permeable” membrane (or filter pad). The retentate is that stream which has been enriched with the solutes of suspended solids which have not passed through the membrane (or filter pads). Water can be continually added to the retentate vessel at the same rate at which the sucrose-rich permeate is leaving in order to avoid over concentration of the solids. Once sufficiently filtered, the retentate, containing the isolated oocysts, can then be stored in any suitable medium and temperature until sporulation. The isolated oocysts may be placed in sterile water and stored at about 2-8° C.

Preferably, the decanter centrifuge method of concentration, described above, is used to concentration the filtrate retained from sieving and the volume of solids obtained from a decanter centrifuge is measured by volumetric measurement. Such measurement may be taken by centrifuging about 50 ml of the concentrated filtrate for about 10 minutes at 1,500×g (r_{average}) in a centrifuge with 50 ml conical tube adapters. Any centrifuge that produces the preferred forces on the filtrate may be used. The percent solids can be calculated by multiplying the volume of the solid by 2. Other well known methods may also be used to calculate solids and can be determined by one skilled in the art. The solids content is preferably then adjusted to less than 60% solids, with domestic water, if necessary. More preferably, the percent solids is brought to below about 50% solids by the addition of domestic water, and most preferably the percent solids is brought to below about 40% solids by the addition of domestic water.

Then a HFCS solution, in a percent volume from about 30% to about 40% of the solid collected from the concentration/centrifugation step, is preferably added. This typically brings the density of the liquid phase up to the point where the oocysts float. The density of the liquid is preferably brought up to at least 1.09 g/ml and can be brought up to an amount higher than 1.09 g/ml. The density of the liquid is preferably between 1.09 g/ml and about 1.20 g/ml, more preferably to be between 1.09 g/ml and about 1.14 g/ml, and most preferably to be between about 1.09 g/ml and about 1.10 g/ml. If the density of the liquid is less than 1.09 g/ml, the oocyst-containing liquid may be remixed with additional HFCS solution until the density is at least 1.09 g/ml. This dense liquid is then poured into vessels proper for centrifuging, the vessels preferably balanced with respect one another in their placement in the centrifuge, and then centrifuged.

Preferably, the dense liquid is centrifuged at a temperature from about 4° C. to about 10° C. The density of the liquid phase can then be measured following the first centrifuge run using methods well known in the art. If the density of the liquid is less than 1.09 g/ml, it is preferable to re-mix the liquid phase and the solid phase and add more high fructose corn syrup solution to obtain a density of 1.09 g/ml or greater. These steps can be repeated if necessary in order to obtain the highest yield of oocysts.

Preferably, to the resuspended oocysts from the concentration step described above, is added a volume of HFCS equal to about 30% to about 40% of the volume of the solid fraction. HFCS is preferably added until the density of the liquid phase is brought up to the point where the oocysts float, a density of about 1.09 to about 1.14 g/ml. The entire suspension of oocysts in the HFCS/domestic water suspension may then be separated from the HFCS (see FIG. 5B, step 12).

The HFCS/domestic water suspension is preferably poured into centrifuge bottles, balanced with respect one another in their placement in the centrifuge, and centrifuged for about 15 minutes at 3750× g (rₘₐₓ) and at a temperature from about 40° C. to about 100° C. The buoyant oocysts float to the top of the suspension while heavier unwanted solids settle to the bottom of the bottles. The solids contained in the supernatant preferably contain no more than about 40% solids. If the percent solids found in the supernatant, measured according to the volumetric method described above, is higher than about 40%, then the density is typically too high and the entire suspension needs to be diluted with domestic water and re-centrifuged.

In using the bottle centrifuge method of centrifuging, a large number of the oocysts typically remain in contact with the bottle near the top of the supernatant. This oocyst-containing material may be freed and returned to the supernatant, for e.g., by swirling the bottles or by using a tool, such as a spatula. This does not disturb the solid phase. The bottles may be swirled by hand at room temperature to remove the crust of oocysts on the bottle. In larger batch sizes the vessel used for centrifuging can be cleaned by those methods familiar to one skilled in the art in order to clean the vessel and recover a higher percentage of oocysts.

The supernatant is then poured off into a vessel. If using the bottle centrifuge, rotating the bottles while pouring helps rinse the oocysts off the sides. The solid fraction can then be discarded. The same centrifuge bottles can then be refilled and the process repeated until all of the dense liquid has been centrifuged. The oocysts are now ready to go to the second concentration step which removes residual sucrose.

Concentrating the Oocysts after Flotation

The liquid fraction from the floatation step described above may then be diluted with domestic water and separated from the HFCS. In this process, the oocysts are preferably concentrated prior to the sporulation step. The concentrated oocysts may then be diluted yet again and held prior to sporulation.

Preferably, the liquid phase recovered from the flotation centrifugation step is first diluted with domestic water till the oocysts sink, and then centrifuged to capture the oocysts in the solid phase (FIG. 5B, steps 15-18) to remove a substantial amount of HFCS. The liquid phase recovered from the flotation centrifugation step may also be first diluted with domestic water till the oocysts sink and the suspension is processed with a hydrocyclone. In using the hydrocyclone, the upper fraction is recovered. In subsequent separation of the HFCS, the concentrated oocyst containing suspension is again diluted with domestic water and transferred to a holding vessel prior to sporulation (FIG. 5B, steps 19-20).

Preferably, the volume of the liquid fraction recovered from the flotation step is measured and a sample is taken to assess oocyst count. Sufficient domestic water is preferably added to lower the density of the supernatant to less than about 1.04 g/ml. This allows the oocysts to sink. The density can be measured following the addition of the domestic water using techniques well know in the arts. If the density is not less than about 1.04 g/ml and/or the oocysts have not sunk, additional domestic water may be added until such density is reached and/or the oocysts sink. The oocyst suspension is then poured into centrifuge bottles and centrifuged for preferably about 10 minutes at 1200×g from about 4° C. to about 10° C. The supernatant may be tested for oocyst presence by counting using a microscope and hemocytometer, and the supernatant discarded if an acceptable amount of oocysts are counted in the supernatant. An acceptable amount of oocysts in the supernatant is typically from about 1% to about 5%, preferably about 2%, of the total oocysts loaded at the beginning of flotation step. More of the mixture from the flotation step is then poured on top of the solid fraction generated by centrifugation. While not necessarily being resuspended, the solid fraction may be loosened somewhat, particularly by inverting the bottle a few times. The resuspended solid fraction suspension may then be centrifuged as before, for about 10 minutes at 1200×g from about 4°0 C. to about 10° C., and the process may be repeated until the flotation step mixture has all been centrifuged.

When using the bottle centrifugation method, at this point, there should be several bottles, each with a solid fraction in the bottom. Note, however, with larger batch size the vessel or vessels may vary with equipment that is of appropriate volume and recovery methods may be determined by one skilled in the art. The solid fractions in the centrifugation vessels are then preferably resuspended by shaking them with a minimal amount of domestic water. The solid fractions are rinsed into one or two of the bottles and the bottles filled and balanced with water if necessary. These bottles are preferably centrifuged one last time as before, for about 10 minutes at 1200×g from about 4° C. to about 10° C. The supernatant is then discarded. Any loose solid fractions that comes out with the supernatant can be ignored. The solid fraction can then be resuspended in a minimal amount of domestic water and stored in a single bottle from about 2° C. to about 5° C. pending sporulation, while freezing should be avoided.

Alternatively, the HFCS in the liquid phase recovered from the flotation step can be remove by filtration using filters with a pore size which excludes the oocysts. When filtration is used, tangential flow is preferred. Tangential flow filtration (TFF) is characterized in that an influent stream is separated into two effluent streams, known as permeate and retentate. The permeate is that fraction which has passed through the “semi-permeable” membrane (or filter pad). The retentate is that stream which has been enriched with the suspended solids which have not passed through the membrane (or filter pads). Once sufficiently filtered, the retentate, containing the isolated oocysts, can then be stored in any suitable medium and temperature until sporulation. Preferably, the isolated oocysts are placed in sterile water and stored at about 2-8° C. Note that the tangential flow filtration is an alternative embodiment to concentrating the oocysts after flotation and TFF at this step should not be confused with TFF used during sterilization.

Alternatively, the volume of the liquid fraction recovered from the flotation step is measured and a sample is taken to assess oocyst count. Sufficient domestic water is added to lower the density of the supernatant to less than about 1.04 g/ml. This allows the oocysts to sink. The density is measured following the addition of the domestic water using techniques well know in the arts. If the density is not less than about 1.04 g/ml and/or the oocysts have not sunk, additional domestic water may be added until such density is reached and/or the oocysts sink. The oocysts suspension may then be processed through a hydrocyclone at a flow rate of about 2 gallons per minute and at a pressure between about 120 psig and about 130 psig.

Batch size and scale can lead one skilled in the art to utilize various centrifugation processes based on batch size while one skilled in the art can also adjust the correct centrifugation speeds based on the batch size.

Sporulation

Sporulation is performed to transform the cleaned and concentrated oocysts into their next life form, the sporulated oocyst (see FIG. 5B, “Sporulation Suite”). Sporulation may be performed in any suitable container, however, a fermentation vessel is preferred in order to best control temperature, dissolved oxygen, pH, and mixing in addition to monitoring these parameters of the sporulation medium. The capacity of the sporulation vessel varies with batch size and can be adequately selected by one skilled in the art. A preferred fermentor is the New Brunswick BioFlow 3000 (available from New Brunswick Scientific Company, Edison, N.J.).

Sporulation is achieved by subjecting the oocysts to an oxidative challenge. In this step, the oocysts are contacted with an oxidizing agent which is effective to promote sporulation but does not result in the death of the oocysts. As described below, the oxidizing agent comprises a principal oxidant other than a source of dichromate. Preferably, the sporulation medium is substantially devoid of potassium dichromate, an alkali metal dichromate, dichromate ions, hexavalent chromium, or other dichromate salts. In a preferred embodiment, the sporulation medium contains not more than about 0.8% by weight of alkali metal dichromate, or not more than about 0.6% by weight of alkali metal dichromate, or not more than about 0.4% by weight of alkali metal dichromate, or not more than about 0.2% by weight of alkali metal dichromate, more preferably not more than about 0.1% by weight of alkali metal dichromate, and most preferably the sporulation medium is substantially free of an alkali metal dichromate. In another preferred embodiment, the sporulation medium contains not more than about 3.0% by weight of dichromate ions. In yet another preferred embodiment, the sporulation medium contains not more than about 1.5% by weight hexavalent chromium.

Oocysts concentrated by the methods and processes of the instant invention and described above are collected over a time period sufficient to create a batch size suitable for a fermentation vessel of desired volume. The collected concentrated oocysts are then deposited into a sporulation vessel, an oxidizing agent is added and sporulation is allowed to occur (see FIG. 5B, step 21). Domestic cold water is used to rinse the container or containers holding the oocysts prior to their contact with the fermentation vessel. The sporulated oocysts and the rinse is then transferred to a separation device (FIG. 5B, step 22). Sporulation may also be achieved by depositing concentrated oocysts in a fermentation vessel, subjecting the oocysts to an oxidative challenge by contacting the oocysts with an oxidizing agent, such as oxygen, or sodium hypochlorite, in an aqueous medium, wherein the percent saturation of dissolved oxygen in the medium is maintained at preferred levels, pH is controlled between preferred levels by the alternative addition of an acid or a base, the suspension is mixed to near homogeneity, and temperature is between preferred temperatures over a preferred period of time. An anti-foaming agent may be added during the sporulation process. Preferably solids do not exceed more than about 50%. Preferably solids are less than about 35%. More preferably still, solids are less than about 25%. Sufficient domestic water is added to the sporulation vessel to achieve this ratio between solid and liquid phase. The liquid phase in the fermentation vessel is termed the sporulation medium.

Preferably, the oxidizing agent used is oxygen. Oxygen may be added in the form of air or as pure oxygen.

Alternatively, sufficient 5.25% sodium hypochlorite is added to the fermentor to achieve the following initial concentrations upon dilution of active chlorine concentrations for the individual subspecies. The values are approximate and indicate preferred maximum concentrations that do not inhibit sporulation:

TABLE 1

| Spp. of Eimeria | weight % |
|---|---|
| E. Acervulina | 0.01 |
| E. Maxima | 0.05 |
| E. Tenella | 0.05 |

During sporulation, the percent saturation of dissolved oxygen content in the aqueous medium is maintained at at least 30% of saturation, preferably at at least 40% of saturation, and more preferably at least 50% of saturation. Percent saturation of dissolve oxygen is controlled, by supplying air or molecular oxygen, to achieve consistent and higher yields of sporulated oocysts.

Preferably, percent saturation of dissolved oxygen is maintained by bubbling air through the mixture at a rate sufficient to meet the above ranges. Pure oxygen may also be bubbled through the mixture to maintain the requisite percent dissolved oxygen. Care should be taken so that the flow of oxygen is not so rapid as to cause foaming. If desired, an anti-foaming agent may be added, such as Antifoam A (available from Sigma-Aldrich, St. Louis, Mo.). Oxygen is added by any means practicable. Oxygen may be added by adding both air when lesser flow rates are needed, e.g., when oxygen consumption is relatively low to peak sporulation, to maintained the preferred percent dissolved oxygen saturation while molecular oxygen may be added when the need is greater, e.g., when oxygen consumption is greatest. Oxygen is preferably added at a flow rate of from about 0.1 to about 2.0 liters of gas per liter of material and more preferably from about 0.3 to about 0.5 liters of gas per liter of material. The flow rate may be kept constant despite a greater need to maintain preferred percent saturation of dissolved oxygen as the gas added may consist of air when less oxygen is needed and may consist of molecular oxygen when more oxygen is needed. The preferred fermentor automatically converts from the addition of air to molecular oxygen as needed while controlling a nearly constant flow rate.

The pH level is preferably maintained from about 7.0 to about 7.7, more preferably from 7.2 to about 7.5, and more preferably still the pH is maintained about 7.4. The pH level of the sporulation medium is controlled by adding an acid or a base. Preferably, either sodium hydroxide (5N) or sulfuric acid (5N) is alternatively added to the sporulation medium as needed to maintain the pH near 7.4. When using a fermentation vessel, the acid and/or the base may be added by using a fermentation vessel's automatically controlled peristaltic pumps on the fermentor.

The temperature of the sporulation medium is controlled throughout sporulation. Oocysts are placed in a sporulation vessel at a temperature from a temperature that substantially avoids freezing to about 43° C.; preferably between about 15° C. to about 38° C.; and more preferably between about 20° C. to 30° C. and more preferably still at about 28° C. ±1° C. It will be apparent to those of ordinary skill in the art that the rate of sporulation is temperature dependent, so that the time required for sporulation will generally be less at higher temperatures.

Throughout the sporulation process, the sporulation medium is mixed. Any suitable method of mixing can be used to mix the sporulation medium to about a homogenous state. The exact method of mixing-varies depending on the container used. For example, when bottles or flasks are used, mixing can be achieved by the use of shakers, or magnetic or mechanical stirrers. When vats or fermentors are used, a mechanical stirrer, such as a paddle stirrer is preferred.

Although sporulation is substantially complete within 12 to 18 hours, removal of the sporulated oocysts prior to about 72 hours decreases viability. Therefore, sporulated oocysts are preferably kept under the above sporulation conditions for a preferred time period to provide a more stable population of sporulated oocysts. The oocysts are preferably maintained in the above conditions for approximately 72 to 120 hours, more preferably for 72 to 110 hours, and more preferably still for 72 to 96 hours, to allow sporulation to occur.

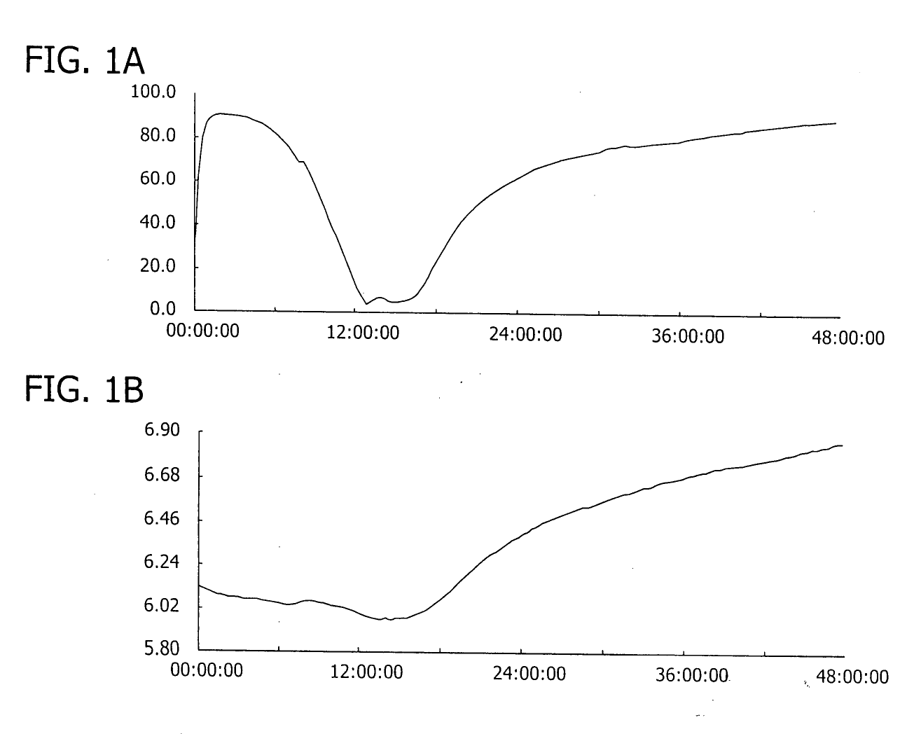

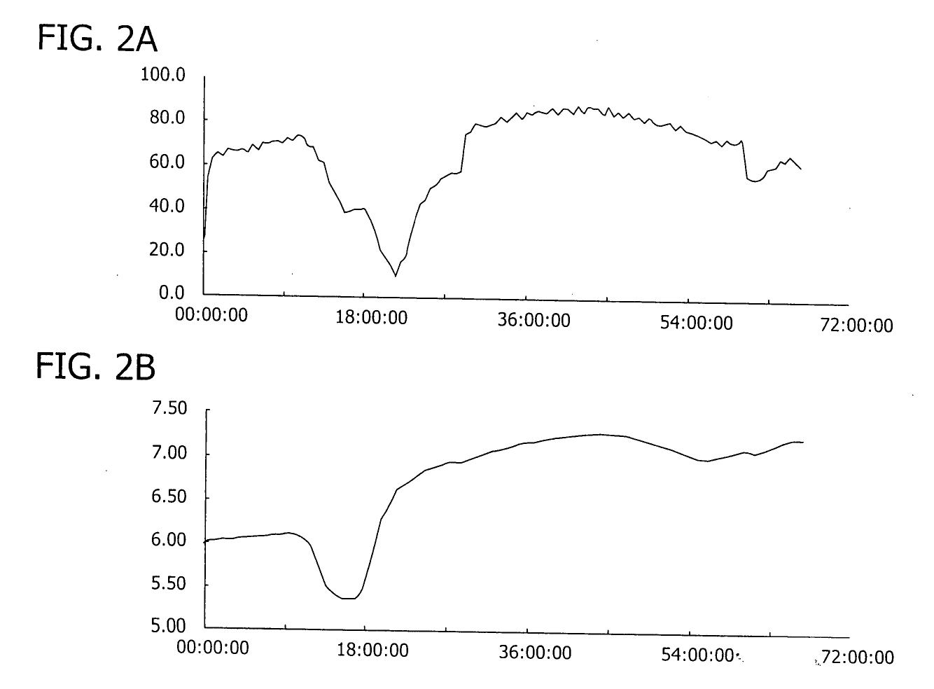

Sporulation start point, end point and rate may be monitored by monitoring: (1) the rate at which oxygen must be added to the sporulation medium to control percent saturated dissolved oxygen; and/or (2) by monitoring the amount of acid or based required to be added to control the pH of the sporulation medium. It has been discovered that sporulation results in an increase in oxygen consumption, as evidenced by a decrease in dissolved oxygen in the sporulation medium, and an increase in pH, that is, if percent saturation of dissolved oxygen and pH are not controlled. When no additional oxygen is added to the sporulation medium, sporulation is indicated by a drop in dissolved oxygen to less than 60% of saturation, more preferably less than 40%, and more preferably still less than 20%. The change in dissolved oxygen can also be measure in terms of percent change. Thus, sporulation can also be indicated by a decrease of at least 10% (i.e., from 50% to 40%), preferably at least 20%, more preferably at least 30%, and more preferably still at least 40% in dissolved oxygen content as expressed in percent of saturation (see FIG. 1A). When pH is not controlled by the alternative addition of an acid or a base, increase in pH of at least about 0.25 pH units, more preferably at least about 0.5 pH units, is indicative that sporulation is occurring (see FIG. 2).

The change in dissolved oxygen and pH do not occur independently. An increase in the oxygen consumption indicates the start point of sporulation. Note, however, that background oxygen consumption will be seen as the sporulation medium is not sterile at this point and so various bacteria will be consuming oxygen as well as the oocysts. However, the increase in oxygen consumption will be significant over the background oxygen consumption so that the sporulation start point, end point, and rate, including peak, are readily ascertainable. A decrease in oxygen consumption indicates a drop in sporulation rate. Once oxygen consumption becomes low and consistent, sporulation is substantially complete, usually after about 18 hours. However, as mentioned above, the sporulated oocysts should be maintained under the sporulation conditions for at least an additional 36 to 48 hours to increase yield. Monitoring of sporulation will assist the practitioner in reaching higher yields of viable sporulated oocysts. Optionally, sporulation can be confirmed by microscopic examination of the oocysts. However, the method of present invention obviates the need for sampling and microscopic examination.

Sterilization

Following sporulation, the sporulated oocysts, are removed from the sporulation vessel, and washed free of the sporulation medium and concentrated by any suitable method, preferably filtration. The entire sterilization process is generally conducted in two phases: (1) contaminants are first removed non-aseptically (see FIG. 5C, steps 23-25); followed by (2) disinfection of sporulated oocysts medium carried out under sterile conditions (see FIG. 5C, steps 26-28). The purpose of this process is to collect sporulated oocysts and filter out contaminants. A further purpose is to concentrate oocysts, preferably by filtration. However, centrifugation may also be used to concentrate the sporulated oocysts. A further purpose is to sterilize the suspension with a disinfectant, for example, sodium hypochlorite (leaving the sporulated oocysts intact), then to remove the disinfectant from and then concentrate the sporulated oocysts. Then, to the sporulated oocysts, is added an appropriate quantities of buffer and antibiotic, preferably phosphate buffered saline (“PBS”) and gentamicin (FIG. 5C, steps 30-31). This sporulated oocysts-containing suspension is then transferred into suitable storage containers for bulk storage prior to final packaging for distribution to consumers.

Separation of the sporulated oocysts from the sporulation medium may be achieved by centrifugal-based separation, such as by bottle centrifuge, decanter centrifuge, or by hydrocyclone. The volume of the batch size will be determinative of the mode of centrifugal-based separation and can be determined by one skilled in the art. The solid fraction from any one of the centrifugal-based separation methods is recovered. If more than about 5% of the oocysts loaded into the centrifugal-based separation unit are in the refuse fraction, a liquid fraction in this embodiment, said fraction is mixed with the solid fraction and recycled through the centrifugal-based separation unit. The recovered solids are then diluted to a volume appropriate for sterilization, preferably by filtration, more preferably by tangential flow filtration.

Preferably, once sporulation is complete, the resultant aqueous suspension of sporulated oocysts is transferred from the fermentation vessel into a receiving vessel of appropriate volume. The transfer of the oocysts from the fermentation vessel is preferably accomplished by using air forced through the fermentation vessel, e.g., pressurizing the headspace, thereby forcing the sporulated oocysts into the awaiting container. A sample of the sporulation medium from the container is then taken to assess sporulated oocyst count and sporulation ratio. Any material still in contact with the fermentation vessel may be removed by using a rinse, e.g., a sufficient amount of domestic water, and the rinse may then be combined with the suspension of sporulated oocyst already transferred from the fermentation container (FIG. 5B-C, step 22). Again, a sample is then taken to assess sporulated oocyst count and sporulation ratio in an effort to ascertain yield. The manner and method in which the sporulated oocysts are harvested from the fermentation vessel varies with the batch size and type of fermentation vessel and may be properly determined by one skilled in the art.

After removal of the suspension of sporulated oocysts from the fermentation vessel, the oocysts are allowed to settle from sporulation medium over a period of several hours, e.g., 8 to 20 hours, while the medium and the oocysts are held at a temperature from a temperature that prevents freezing to about 10° C., more preferably from about 2° C. to about 6° C., and most preferably about 4° C. Sporulated oocysts settle to the bottom of the storage container while contaminants remain suspended or dissolved in the aqueous layer. Preferably, the supernatant is decanted, poured or pumped off using a small peristaltic pump or other method suitable to the volume being removed. Domestic water is then added at a sufficient volume to resuspend the sporulated oocysts. Settling can be carried out in the receiver or in a separate settling vessel.

The sporulated oocysts, now resuspended after collection from settling, are then separated from the sporulation medium. Preferably, separation is by filtration. However, any appropriate means may be used to separate the sporulated oocysts from the sporulation media. More preferably, the filtration process is by means of tangential flow filtration. Tangential flow filtration (“TFF”) is used in this procedure to separate sporulated oocysts from other material that may be present in the suspension, e.g., grit, other microorganisms, etc. In addition to the filter membrane, two essential parts of the TFF system are a retentate vessel, which holds the sporulated oocysts, and a low shear pump that circulates the retentate through the membranes and back into the retentate vessel. The oocysts are retained in the retentate while the permeate is discarded.

The pore size of the filter membrane should be small enough so that sporulated oocysts cannot enter the pores, but large enough to allow bacteria to pass through. In one embodiment, the filter has a pore size of approximately 10-microns. In yet another embodiment the filter has a pore size of approximately 5-microns. A preferred filtration unit is a Consep membrane unit manufactured by North Carolina SRT (available from North Carolina SRT, Inc., 221 James Jackson Ave., Cary, N.C. 27513). However, other filtration units may be used, such as those produced by Millipore (available from Millipore Corporation, 80 Ashby Road, Bedford, Mass. 01730). A preferred filter is the Spectra/Mesh polyester filters (Spectrum Laboratories, Inc., Rancho Dominguez, Calif.; cat no: 146524). Tangential filtration units such as an OPTISEP CL, OPTISEP, or CONSEP may be used, also available from North Carolina SRT. Throughput can be increased by utilizing a larger scale filtration unit. One skilled in the art will recognize that the type of filtration unit needed depends on the volume of the sporulated oocyst suspension. In one, embodiment, an OPTISEP CL unit is used to run about a 1L sporulated oocyst suspension. In another embodiment, a CONSEP unit is used to run about a 10L sporulated oocyst suspension.

When a filtration process is applied to a sporulated oocyst-containing medium the permeate is discarded. Water is added as permeate is removed if the filtration process is conducted at a constant volume. Filtration can be accomplished by gravity flow or by the use of a pump, for example, a peristaltic pump. In a preferred embodiment, the mixture is pumped tangentially over the filter. If a pump is used, the rate of pumping varies with such well known factors as the surface area of the filter, the path length, the total area of the flow channel, and the pore size. Optimum pumping rates can be determined by one of ordinary skill in the art without undue experimentation as such flow rates are a function of surface area of the filtration unit and solids content.

In a further embodiment, the inlet and the outlet tubing for the tangential flow unit are placed into a vessel containing the sporulated oocysts while the permeate tubing is placed in a separate vessel. The pump, for example, a diaphragm pump, is then started to begin filtration. A preferred flow rate is about 1 LPM per 160 cm². The pump rate may also be expressed in terms of lineal velocity. Lineal velocities may be between 20 and 50 centimeters per second. A preferred lineal velocity when using a CONSEP filtration unit is 28 centimeters per second. The pump may be kept running to maintain the flow rate throughout the process. The permeate is sampled and, using a glass slide, observed for sporulated oocysts. The optical density of the permeate sample is also measured using a spectrophotometer at 600 nm (OD₆₀₀). Circulation of retentate over the filter medium is continued if the concentration of oocysts in the permeate has not increased to or exceed a maximum tolerable level. An acceptable concentration is from about not more than 5% of the total sporulated oocysts loaded into the filtration unit. If the sporulated oocysts concentration measures to or above the maximum acceptable level, filtration is stopped. The permeate is recycled and mixed with the retentate and filtration is resumed. Filtration is stopped when the measured OD₆₀₀ is about less than about 0.5 at a lineal velocity of about 28 centimeters per second. However, filtration may be stopped when the measured OD₆₀₀ is about 0.6, again at a lineal velocity of about 28 centimeters per second. Once the desired OD is reached, oocysts from the membranes and the tubing are transferred to the retentate vessel and any oocysts remaining in the membranes and tubing are then flushed with water into the retentate vessel.

The retentate, containing the concentrated oocysts, is then placed in a vessel under refrigeration at about 4° C. from about 15 to about 24 hours. This allows the sporulated oocysts to settle in bottom solid phase and a liquid phase will normally form above the solid phase. The liquid phase is then substantially removed after refrigeration to reduce unwanted volume, e.g., by decanting, pumping, or siphoning. In an alternative embodiment, the retentate is processed immediately after filtration. However, it is preferred to let the retentate rest as described above.

Once the sporulated oocysts have been concentrated by filtration they can be sterilized by means of a chemical disinfectant or sterilizing agent. Preferably, the disinfectant or sterilizing agent is other than an alkali metal dichromate, soluble dichromate moieties, dichromate ions, or potassium dichromate. Sterilization processes are conducted in sterile environments. Sterilization may be accomplished within the filtration device used to concentrate the sporulated oocysts. Alternatively the retentate containing the sporulated oocysts can be washed from the filter and sterilization is accomplished in a vessel separate from the filtration device. Any filtration unit used to sterilize the sporulated oocysts should be sterilized prior to the addition of the unsterilized sporulated oocysts. The filtration unit is sterilized by autoclaving. Alternatively, the filtration unit is sterilized by passing steam at approximately 250° C. through the system for at least about 30 minutes at approximately 20 psi. Alternatively, the unit is chemically sterilized by treating the system with 5% sodium hypochlorite for at least about 10 minutes wherein the sodium hypochlorite contains at least about 5% available chlorine by weight.

The agent used for sterilizing the sporulated oocysts preferably is one which kills bacteria and viruses, but does not kill the sporulated oocysts. Preferably, the disinfectant used kills the infectious bursal disease virus (IBDV), chick anemia (CAV) viruses, and related bacteria. As IBDV is known to be a robust virus, a sterilization agent that kills IBDV will kill other, less robust microorganisms as well. An agent that eliminates IBDV is considered to substantially eliminate microorganisms.

One example of such a disinfectant is sodium hypochlorite. Other sterilization agents or disinfectants are well known in the art. The concentration of disinfectant used varies with the agent chosen to accomplish sterilization. For example, sodium hypochlorite may be used at a concentration preferably in the range from about 1% to about 10%, and more preferably in the range of about 2% to about 5% wherein the percent represents the percent of available chlorine by weight. The time during which the sporulated oocysts are exposed to the disinfectant varies depending upon factors such as the concentration of the disinfectant and the volume of the batch of sporulated oocysts. For example, the sporulated oocysts may be treated with approximately 5% sodium hypochlorite, wherein the percent represents the percent of available chlorine by weight, from about 2 to about 20 minutes, more preferably from about 5 to about 18 minutes, and most preferably for about 10 minutes.

Once the filtration unit is sterilized, the vessel holding the sporulated oocyst suspension is then removed from refrigeration. The clear upper layer is removed by pumping, pouring, or suctioning off the supernatant, leaving the bottom sporulated oocyst fraction. The latter fraction is then transferred to a retentate vessel of adequate volume. The previous vessel is then rinsed with domestic water. The domestic water rinse is then added to holding vessel and stirred by adequate means. In one embodiment, stirring is accomplished by means of a magnetic stir bar while the retentate vessel is sitting on a magnetic stirrer. In another embodiment, stirring is accomplished by means appropriate for the volume of the retentate vessel, such as with a paddle.

Next, a volume of disinfectant (e.g. about 10% aqueous sodium hypochlorite solution) that is approximately equal to the volume of suspension in the retentate vessel is added to the sporulated oocyst suspension. The sporulated oocyst suspension containing the sodium hypochlorite should have a solids content of preferably less than about 30%, more preferably to less than about 25%, and even more preferably to less than about 15%, and most preferably to less than 7.5%. A solids content of less than about 7.5% is preferred as the reduced solids content produces a higher assurance of sterility. When solids are brought to about 15%, there is approximately 5% sodium hypochlorite, wherein the percent represents the percent of available chlorine by weight, in the suspension. Alternatively, the solids concentration is adjusted to about 15% prior to sodium hypochlorite addition. After this addition, the solids are 7.5% and the sodium hypochlorite is at 5%. Either way, the suspension is mixed thoroughly by adequate means and, in one embodiment, allowed to stand for preferably 5 minutes, more preferably 8 minutes, and most preferably about 10 minutes. Standing time will vary depending on batch size and volume and may be adjusted as needed. However, standing may be avoided and filtration may occur immediately after dilution.

The retentate vessel containing the sporulated oocysts suspension is then connected to the filtration unit. Autoclaved or otherwise sterile water is used as a water source during the sterilization process. The retentate pump is then activated while the permeate line is pinched or clamped closed. Once any air bubbles have been substantially eliminated from the membranes and tubing the permeate line may be opened and directed to a collection vessel outside a sterile environment. Permeate is then analyzed to verify that there is no significant loss of oocysts via the permeate.

In an alternative to using filtration to remove the disinfectant from the oocyst suspension, centrifugation may be utilized.

While the disinfectant-rich permeate leaves the filtration unit, sterile water is added to replenish the volume. Filtration is continued and water is added as needed to control the percent solids during filtration. A sample is then taken from the permeate to determine total chlorine level. Chlorine level can be detected using CHEMetrics Vacuette Kit, available from CHEMetrics, Inc., Route 28, Calverton, Va., 20138. The total level of chlorine should be reduced to less than about 1 ppm by filtration. When the permeate contains less than about 1 ppm of the chlorine the retentate, which contains the oocysts, also contains less than about 1 ppm. Once the desired level of chlorine is reached, filtration may be continued without adding more water to reduce the overall volume of the sporulated oocysts suspension.

When the desired retentate volume is reached, the pump feed tubing is removed from the retentate vessel and placed into a vessel containing sterile water and filtration is continued. Once substantially all of the suspension has been flushed out of the tubing and the filter housing, the pump is shut off and filtration is complete.

As described above, the vaccine may be filtered at one or more step of production by tangential flow filtration. When tangential flow filtration is used, the pore size of the filter membrane is preferably small enough so that sporulated oocysts cannot enter the pores, but large enough to allow bacteria to pass through. Tangential flow filtration thus substantially removes bacterial contaminants from an oocyst preparation. Thus, the composition of the present invention is preferably characterized as substantially free of bacterial contamination. Furthermore, since filtration (such as tangential flow filtration) may be used at multiple steps of production, bacterial contaminants may be removed from the composition at several different points during production. Thus, bacterial contaminants may be removed from the composition at one or more step of production, preferably by filtration, and most preferably by means of tangential flow filtration.

Preferably, the vaccine composition of the present invention comprises not more than limited amounts of alkali metal dichromate, hypochlorite ion, chloramine, and/or hydrogen peroxide. Thus, for example, the vaccine composition may contain not more than about 0.8% by weight of alkali metal dichromate, or not more than about 0.6% by weight of alkali metal dichromate, or not more than about 0.4% by weight of alkali metal dichromate, or not more than about 0.2% by weight of alkali metal dichromate, or not more than about 0.1% by weight of alkali metal dichromate. In a preferred aspect, the composition may be characterized as being substantially free of alkali metal dichromate. In addition, the composition preferably contains not more than about 3.0% by weight of dichromate ion or not more than about 0.15% by weight of hexavalent chromium.

Furthermore, the composition preferably contains not more than about 10 parts per million hypochlorite ion, or not more than about 5.0 parts per million of hypochlorite ion, or not more than about 2.5 parts per million of hypochlorite ion, or not more than about 1.0 part per million of hypochlorite ion, or not more than about 0.5 parts per million of hypochlorite ion. Advantageously, the vaccine composition may be characterized as substantially free of hypochlorite ion.

In another aspect, the composition preferably contains not more than about 0.75% chloramine by weight, more preferably, not more than about 0.50% chloramine by weight, or not more than about 0.25% chloramine by weight, or not more than about 0.01% chloramine by weight. Advantageously, the vaccine may be characterized as substantially free of chloramine.

Additionally, the composition may preferably contain not more than about 1000 mg/l hydrogen peroxide, or not more than about 500 mg/l hydrogen peroxide, or not more than about 250 mg/l hydrogen peroxide, or not more than about 100 mg/l hydrogen peroxide. Preferably, the vaccine is characterized as substantially free of hydrogen peroxide.

Furthermore, the composition preferably contains not more than about 0.75% chloramine by weight, not more than about 10.0 ppm hypochlorite ion, not more than about 0.8% by weight of alkali metal dichromate ion, and not more than about 1000 mg/l hydrogen peroxide.

It is desirable, though not essential, that the composition be characterized as being substantially free of chloramine, sodium hypochlorite, other alkali metal hypochlorite, hypochlorite ion, alkali metal dichromate, dichromate ion, hexavalent chromium in any form, and hydrogen peroxide.

The pH of the composition of the present invention is preferably from about 4.0 to about 8.0, and more preferably from about 7.2 to about 7.6. Most preferably, the pH of the composition is about 7.4.

The density of the compositions of the present invention may vary. However, the typical density of the compositions is from about 0.97 to about 1.03, preferably from about 0.99 to about 1.01 g/ml. The difference in density between the sporulated oocysts and the medium in which they are suspended is preferably not more than 3%, and more preferably not more than about 1% based on the density of the medium.

The viscosity of the compositions of the present invention varies depending on temperature and the presence or absence of thickening agents, but is approximately that of water. For example, the viscosity of the compositions at 25° C. may be from about 0.75 to about 1.90 centipoise, preferably from about 0.79 to about 1.00 centipoise. In an alternative embodiment, the viscosity may be from about 1.31 to about 1.81 centipoise.

Furthermore, the compositions of the instant invention may contain at least about 1.5×10⁴ viable sporulated oocysts per milliliter. Preferably, said composition contains not more than about 0.75% chloramine by weight, not more than about 10.0 ppm hypochlorite ion, not more than about 0.8% by weight of alkali metal dichromate, and not more than about 1000 mg/l hydrogen peroxide. Preferably, the composition contains at least about 1.5×10⁴ viable sporulated oocysts per milliliter, and is substantially free of bacterial contamination. Advantageously, the composition contains at least about 1.5×10⁴ viable sporulated oocysts per milliliter, and bacterial contaminants have been removed from the composition.

The vaccine may be concentrated, requiring dilution before administration, or the vaccine may be ready for administration. The concentrated vaccine may be diluted with any suitable diluent to concentrations suitable for various forms of administration. Any form of vaccine administration known in the art may be used, including, but not limited to, intra-yolk sac injection administration, per Os, in ovo administration, orally, e.g. oral gavage, delivery via spray cabinet, or top-fed via spray on to food, such as OASIS® Hatchling Supplement; topically, e.g. spraying; and parenteral routes, e.g. subcutaneous, intramuscular or intraperitoneal injection. The composition may be administered to an animal, such as a member of the class Aves, to treat and prevent coccidiosis.

Such methods for administration are well known in the art. For example, methods for in ovo administration of vaccines are described in, for example, WO 96/40234, herein incorporated by reference. Methods for administration of materials by intra yolk sac injection using either a needle or syringe or an automatic injection device are described in U.S. Pat. No. 5,311,841, herein incorporated by reference. Briefly, the composition to be administered is introduced into the yolk sac through a hypodermic needle inserted in an area having a diameter of about 1 cm on the ventral surface of the chick with the navel being located approximately half-way between the center of the target area and its 12 o' clock position. In the preferred embodiment, administration of the preparation is accomplished within about 48 hours of hatching. The volume administered can vary, but preferably contains a sufficient number of coccidial organisms to result in an immune response. As will be recognized by those in the art, the exact volume administered will vary with well known factors, such as, the concentration of coccidial organisms in the composition, the accuracy of the injection equipment used, and the size of the animal. In general, the volume administered should be large enough so that it can be accurately measured, but not so large as to cause injury to the animal. For intra yolk sac administration, injection volumes are preferably between 0.01 ml and 5 ml, more preferably between. 0.025 ml and 1 ml and more preferably still between 0.05 ml and 0.5 ml. Other methods of administering vaccines have been described herein, or are known in the art.

Preferably, the oocysts of the compositions of the present invention include sporulated oocysts. The number of sporulated oocysts present in the compositions will preferably comprise the minimum number of sporulated oocysts required to comprise an effective dose for immunizing purposes. Preferably, each species is present in such number that the population of that species constitutes at least a minimum immunizing dose. Immunizing doses may differ depending on the route of administration. For oral administration, e.g., either oral gavage or oral consumption, a preferred immunizing dose comprises at least about 100 viable sporulated oocysts (v.s.o.) of Eimeria tenella and Eimeria acervulina and at least about 50 v.s.o. of Eimeria maxima per dose. Such preferred minimum immunizing dose contemplates some degree of “cycling,” wherein the administered protozoa complete an entire life cycle and pass through the chick, are shed as unsporulated oocysts, sporulate, and are re-ingested by the organisms. Depending on a number of conditions, such as sporulation conditions and coccidial protozoa present in the rearing facility apart from the vaccine composition and to which organisms are exposed, the minimum immunizing dose may vary. For spray applications, intra-yolk sac administration, and in ovo administration, the preferred immunizing dose differs and can be determined by one skilled in the art without undue experimentation, and is dependent on a number of factors, including but not limited to the species of coccidial protozoa in the vaccine, “cycling,” and the conditions in which the poultry is kept. The minimum immunizing dose may differ from one production facility or geographic location to the next. Preferably, the minimum immunizing dose, including the proper identification of the species of coccidial protozoa present in the vaccine and in the production facility, are determined so that an effective vaccine composition can be formulated. The preferred oral dosage disclosed herein is a useful benchmark for oral minimum immunizing dosages but may be adjusted as necessary in order to confer an immunological benefit without significantly impacting performance. Various other minimum immunizing doses are known in the art and may provide alternative benchmarks for practice of the invention.

In one example, the dosage is from about 100 to about 1000 viable sporulated oocysts (“v.s.o.”) per dose. The vaccine compositions may be provided in units of doses. For example, the compositions may be provided in 10 milliliter vials, with 1000 doses per vial. Thus, for example, in an embodiment when the dosage is about 100 v.s.o. per dose, the composition preferably contains at least about 10,000 v.s.o. per milliliter. In another embodiment, when the dosage is about 500 v.s.o. per dose, the composition preferably contains at least about 50,000 v.s.o. per milliliter. In a further embodiment, when the dosage is about 1,000 v.s.o. per dose, the composition preferably contains at least about 100,000 v.s.o. per milliliter.

The compositions typically contain at least about 10,000 oocysts per milliliter, may contain at least about 50,000 oocysts per milliliter, or may contain at least about 100,000 oocysts per milliliter.

Preferably, the compositions comprise not more than about 10× the minimum immunizing dose (MID) per unit dose of viable sporulated oocysts, sporocysts, sporozoites, or any combination thereof. It is further preferred that the compositions comprise not more than about 9× the MID, or not more than about 8× the MID, or not more than about 7× the MID, or not more than about 6× the MID, or not more than about 5× the MID, or not more than about 4× the MID, or not more than about 3× the MID, or not more than about 2× the MID per unit dose. The compositions may also comprise not more than about 1× the MID per unit dose.

The compositions may comprise from about 1× to about 10× the MID of viable sporulated oocysts, sporocysts, sporozoites, or any combination thereof, from about 1× to about 8× the MID, or from about 1× to about 5× the MID, or from about 1× to about 3× the MID per unit dose. In certain exemplary embodiments, the compositions comprise about 1× the MID of viable sporulated oocysts, sporocysts, sporozoites, or any combination thereof, about 2× the MID, or about 3× the MID, or about 4× the MID, or about 5× the MID, or about 6× the MID, or about 7× the MID, or about 8× the MID, or about 9× the MID, or about 10× the MID per unit dose.

The vaccine may be diluted with a pharmaceutically acceptable carrier, diluent, or excipient prior to administration, for example, from 10 milliliters to about 250 milliliters, or from about 10 milliliters to about 2.5 L, or from about 10 ml to about 4.0 L. Such diluted vaccines are preferably sterile and comprise viable sporulated oocysts of at least one species of protozoa known to cause coccidiosis, and a pharmaceutically acceptable carrier, diluent, or excipient. It will be understood the starting volume of 10 ml is convenient, but primarily exemplary. Dilutions of 25 to 1, 25-2500 to 1, 25-4000 to 1, or 2500-4000 to 1 can readily be effected using any select starting volume.

The diluted vaccine may comprise at least about 1,000 oocysts per milliliter. Preferably the diluted vaccine is substantially free of bacterial contamination. Bacterial contaminants are preferably removed from the diluted vaccine. The diluted vaccine may comprise at least about 1,000 oocysts per milliliter, not more than about 0.75% chloramine by weight, not more than about 10.0 ppm hypochlorite ion, not more than about 0.8% by weight of alkali metal dichromate, and not more than about 1000 mg/l hydrogen peroxide, and is preferably characterized as substantially free of alkali metal dichromate, hypochlorite ion, chloramine, and hydrogen peroxide.

In an exemplary embodiment, the instant invention may be a concentrated vaccine ready for dilution and then administration wherein said concentrated vaccine contains at least about 10,000 viable sporulated oocysts per milliliter, and is characterized as substantially free of alkali metal dichromate, hypochlorite ion, chloramine, and hydrogen peroxide.

The vaccine composition of the present invention may comprise viable sporulated oocysts containing less than about 5.0×10⁻³ μg of alkali metal dichromate per oocyst, preferably less than about 3.8×10⁻¹ μg of alkali metal dichromate per oocyst, or less than about 1.3×10⁻³ μg of alkali metal dichromate per oocyst, or less than about 6.3×10⁻⁵ μg of alkali metal dichromate per oocyst. Even more preferably, the vaccine composition may be characterized as substantially free of alkali metal dichromate.

The compositions of the present invention are preferably sterile. For the purposes of the present invention, sporulated oocysts, sporocysts, or sporozoites and compositions containing sporulated oocysts, sporocysts, sporozoites, or any combination thereof are considered sterile if samples of liquids containing the oocysts do not have detectable amounts of live or viable bacteria, viruses, or fungi. In particular, it is preferred that the compositions be substantially free of Salmonella, infectious bursal disease virus (“IBDV”) and chick anemia virus (“CAV”). Detection of live bacteria, virus, or fungi can be accomplished by any method known in the art, such as the methods described in U.S. Application Pub. No. US2002/0160022 Al, or U.S. application Ser. No. 09/708,918, herein incorporated by reference in their entirety.

The compositions of the present invention may further comprise a pharmaceutically acceptable carrier, diluent, or excipient. Sporulated oocysts may be diluted with a pharmaceutically acceptable carrier, diluent or excipient to the desired concentration. Preparations can be formulated according to the known art using suitable dispersing or wetting agents and suspending agents. The preparation may be an injectable solution or suspension in a nontoxic parenterally acceptable diluent or solvent. Among the acceptable vehicles and solvents that may be employed are water, Ringer's solution, and isotonic sodium chloride solution. The vehicles and solvents used may optionally include a buffering agent, such as phosphate buffer. Other sterile, conventionally employed solvent or suspending medium may also be used. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful. Other possible formulations will be apparent to those skilled in the art. Formulation of drugs is discussed in, for example, Hoover, John E., Remington's Pharmaceutical Sciences, Mack Publishing Co., Easton, Pa (1975), and Liberman, H. A. and Lachman, L., Eds., Pharmaceutical Dosage Forms, Marcel Decker, New York, N.Y. (1980).

The compositions of the present invention may also comprise a variety of additives. For example, post-challenge performance improvement compositions, agents that ameliorates a decrease in post-challenge performance, thickening agents, growth stimulants, and oxidizing agents may optionally be added to the compositions of the present invention. Such additives are known to those skilled in the art, and can be found, for example, in Plotkin and Orenstein, Vaccines, Third Ed., W. B. Saunders, 1999; Roitt et al., Immunology, Fifth Ed., Mosby, 1998; and Brostoff, et al., Clinical Immunology, Gower Medical Publishing, 1991. Such additives are also described in U.S. application Ser. No. 10/728,194.

The preparation can also contain or be administered in conjunction with at least one nutrient, for example and without limitation, vitamins, minerals, amino acids, sugars and fatty acids. When administered in conjunction with the preparation, the nutrient can be administered at the same time, at a time prior to, or at a time after administration of the preparation. When administered at the same time as the preparation, the nutrient can be contained in the preparation or be administered separately. Administration of the nutrient can be by any suitable means known in the art, including, but not limited to oral and parenteral routes.

Although not necessary, a buffering agent may also be added to the diluent. Buffers are utilized in the storage of vaccine compositions as they prolong viability compared to the use of sterile water or sterile water containing gentamicin. Many suitable buffers are known in the art including, but not limited to, phosphate buffer, bicarbonate buffer, citric acid and tris buffers. The diluent may, for example, comprise 0.5× phosphate buffered saline (PBS). Preferably a volume of buffer is used that results in a concentration of sporulated oocysts suitable for transfer to containers that are ultimately used by the consumer as a vaccine for the prevention of coccidiosis.

The diluent may also optionally comprise a bactericide or other preservative. Any bactericide that is suitable for use in pharmaceutical compositions, and especially compositions that are administered to food animals, can be used. Non-limiting examples of bactericides include potassium perchlorate, hypochlorous acid, sodium hydroxide and antibiotics. Preferred concentrations of chemical bactericides in final concentration in the vaccine, include: from about 0.10 wt % to about 0.75% potassium perchlorate, from about 1 ppm to about 5 ppm hypochlorous acid, and from about 0.5 mM to about 1.5 mM sodium hydroxide. In another aspect of the instant invention, any antibiotic which is suitable for incorporation into compositions to be administered to animals, and especially food animals can be used. In one embodiment, the antibiotic is gentamicin. A preferred concentration of gentimicin is from about 20 to about 30 μg/ml, more preferably about 30 μg/ml.

Thus, a buffer, such as PBS, and a bactericide may be added to the compositions of the present invention. For example, 1×PBS containing 60 μg/ml gentamicin may be added in a 1:1 ratio to a disinfected sporulated oocyst suspension to result in a suspension of sporulated oocysts in 0.5×PBS with 30 μg/ml gentamicin. The suspension may then stored under refrigeration, preferably around 40° C. for future use. Alternatively, 1×PBS may be added to the sporulated oocyst suspension while no bactericide is added to the sporulated oocyst suspension.

The compositions of the present invention preferably comprise viable sporulated oocysts, sporocysts, sporozoites, or any combination thereof. The use of viable sporulated oocysts, sporocysts, or sporozoites is important to ensure protection against Eimeria strains, although those skilled in the art will appreciate that vaccines may also be formulated using killed oocysts, sporocysts, or sporozoites. For purposes of the present invention, the compositions preferably comprise a sufficient amount of viable sporulated oocysts, sporocysts, sporozoites, or combination thereof, of each species, so that the composition contains at least a minimum immunizing dose of each species of viable sporulated oocysts, sporocysts, or sporozoites contained in the composition.

The Eimeria strains used in the compositions of the present invention may also be coccidiostat sensitive, have consistent viability and infectivity, and have broad immunogenicity, while causing minimum reaction in vaccinated birds. Furthermore, the strains used in the compositions of the present invention will preferably have broad immunogenicity, and will be able to, for example, protect against a challenge from a variety of different strains of E. maxima. For example, the strains used in the compositions of the present invention may be those described in U.S. Provisional Application Ser. No. 10/728,194.

Given the sensitivity of these Eimeria strains to various anti-coccidial drugs, it is preferable that anti-coccidial drugs are not administered within 7 days, more preferably within 14 days, and even more preferably within 21 days post-vaccination with a composition of the present invention.

In formulating vaccines, vaccine safety and reactions to vaccination may be taken into account. The process of immunization for coccidia is like a sub-clinical case of the disease. This results in activation of the immune system, which pulls nutrients away from growth. Ideally, this should be hardly noticeable if the dose is well controlled. The compositions of the present invention preferably result in no significant performance setback due to impaired feed conversion, depressed growth or mortality. The performance and safety of a vaccine may be determined by measuring the body weight, feed efficiency, performance index, weight gain, feed to gain ratio, livability, and/or lesion scores of vaccinated animals in comparison to non-vaccinated animals.

A composition of the present invention preferably comprises viable, oocysts, sporocysts, sporozoites, or any combination thereof, of at least one species of protozoa known to cause coccidiosis, and a pharmaceutically acceptable carrier, diluent, or excipient. Such composition is preferably sterile and substantially free of bacterial contamination, and the oocysts, sporocysts, and/or sporozoites are preferably wild type, or derived from a wild type strain. Preferably, the composition comprises sporulated oocysts. Advantageously, the composition comprises not more than about 10× the minimum immunizing dose per unit dose of said sporulated oocysts. The composition may also comprise at least about 10,000 oocysts/ml.

The composition of the present invention may also comprise viable, oocysts, sporocysts, sporozoites, or any combination thereof, of at least one species of protozoa known to cause coccidiosis, and a pharmaceutically acceptable carrier, diluent, or excipient. Preferably, the composition is sterile, the oocysts, sporocysts, and/or sporozoites are wild type or derived from a wild type strain, and bacterial contaminants have been removed from the composition at one or more step of production. Even more preferably, the composition comprises sporulated oocysts. The composition may comprise not more than about 10× the minimum immunizing dose per unit dose of said sporulated oocysts. The composition may also comprise at least about 10,000 oocysts/ml.

The present invention also provides a composition comprising viable, oocysts, sporocysts, sporozoites, or any combination thereof, of at least one species of protozoa known to cause coccidiosis and a pharmaceutically acceptable carrier, diluent, or excipient. Preferably, the composition comprises not more than about 0.75% chloramine by weight, not more than about 10.0 ppm hypochlorite ion, not more than about 0.8% by weight of alkali metal dichromate, and not more than about 1000 mg/l hydrogen peroxide. Advantageously, the composition is sterile, and the oocysts, sporocysts, and/or sporozoites are wild type or derived from a wild type strain. The composition preferably comprises sporulated oocysts. The composition may be substantially free of bacterial contamination, and/or bacterial contaminants may have been removed from the composition at one or more step of production. The composition advantageously comprises not more than about 10× the minimum immunizing dose per unit dose of said sporulated oocysts. The composition may also comprise at least about 10,000 oocysts/ml.

Surfactants

Surfactants, also known as surface active materials, are used herein to prevent or reduce the aggregation of oocysts, sporocysts, and/or sporozoites. Surfactants are a large group of materials which act to reduce the tension at the point of contact between phases. Surfactants can include materials derived from a natural source, as well as a variety of synthetic materials.

Surfactants are typically amphiphilic, and are characterized by a hydrophilic head and a hydrophobic tail region. Surfactants have a high affinity for interfaces, such as the interface between phases (e.g. air/water interface; solid/water interface). At an interface, the surfactant can typically align itself so that the hydrophilic head and the hydrophobic tail may each be in contact with a favorable environment (e.g. the hydrophobic tail is pushed away from water). If the concentration of surfactant is great enough, this action will result in a layer of surfactant adsorbing onto the walls of the containing vessel, and/or accumulating at the air/solution interface. The surfactant may also form micelles, or attach its hydrophobic tail to other hydrophobic objects.

Surfactants may act to prevent and reduce aggregation of oocysts, sporocysts, and/or sporozoites by more than one mechanism. Without wishing to be bound by any particular theory, it is believed that certain areas on oocysts are hydrophobic, and are thus attracted to the hydrophobic areas on other oocysts, to interfaces, and to other hydrophobic areas, much like the hydrophobic moiety of surfactants. This attraction results in aggregation of oocysts at vaccine/composition-air or vaccine/composition-surface interfaces, and in the sticking of oocysts to each other. However, when certain surfactants are added to a composition containing oocysts, such as a vaccine, it is believed that the hydrophobic moiety of the surfactant sticks to either the hydrophobic part of the oocyst, or to an interface, such as the air/composition interface the composition/container (e.g. glass) interface, or the composition/cap interface (for example, when the container cap or stopper becomes wetted), as described above. This in turn either reduces or prevents the aggregation of oocysts. A similar mechanism may occur with respect to sporocysts and/or sporozoites.

Applicants have found that certain surfactants are effective at reducing and preventing oocyst, sporocyst, and/or sporozoite aggregation. As previously mentioned, a variety of both natural and synthetic materials may act as a surfactant. In one instance, the surfactant may be derived from a natural source, including for example, various proteins, amino acids, peptides, casein (e.g. β-casein), serum albumin, and various gums. In particular, casein and serum albumin have been found to be effective at decreasing and preventing aggregation of oocysts. It is generally preferable that any naturally derived surface active materials used in combination with the compositions and methods of the present invention are not derived from animals. Rather, if a surface active material which is ordinarily derived from an animal (e.g. casein, serum albumin, etc.) is to be used, it is preferable that that surface active material be synthetically produced.

Naturally derived surfactants are generally very large compared to synthetic surfactants, and may contain only a very small hydrophobic portion. Because of their large size and mass, a much larger quantity of such materials must typically be used in comparison to the quantity of synthetic surfactant needed to achieve the same results.

Preferably, the surfactants used herein are synthetic. Although, as previously indicated, some naturally derived surfactants may be synthetically produced, the term synthetic surfactants typically refers to non-naturally derived materials. Such synthetic surfactants generally have a lower molecular weight (e.g. typically less than about 1500) than naturally derived surfactants. A variety of synthetic surfactants are known in the art. In general, synthetic surfactants may be classified as anionic, cationic, or non-ionic surfactants, among others. Preferably the surfactants used herein are non-ionic surfactants and/or anionic surfactants, and most preferably are non-ionic surfactants.

Non-ionic surfactants may include a variety of compounds, such as amides, esters, semipolar, and zwitterionic compounds, polyethylene glycol ethers, and polyoxyethylene compounds, among others. Particularly preferred are the non-ionic surfactants Tween-20 (i.e. polyoxyethylenesorbitan monolaurate), Tween-80 (i.e. polyoxyethylenesorbitan monooleate), Triton X-100 (i.e. t-octylphenoxypolyethoxyethanol), Triton X-200, Tergitol 15-S-9 (alkyloxypolyethyleneoxyethanol), and Tergitol 15-S-12 (alkyloxypolyethyleneoxyethanol). Other surfactants, such as those identified by the methods described herein, may also be used to prevent and reduce aggregation.

Combinations of two or more surfactants may also be used in the compositions and methods of the present invention to prevent and reduce aggregation. In general, any combination of surfactants effective at reducing and preventing aggregation may be used herein. In a preferred example, two or more non-ionic surfactants, such as those described above, are used in combination. Alternatively, one or more non-ionic surfactant may be used in combination with, for example, one or more anionic surfactant and/or one or more naturally derived surfactant. As will be apparent to one skilled in the art, numerous other combinations of surfactants may also be effective.

As previously indicated, surfactants constitute a broad group, and may have varying degrees of hydrophobicity. Consequently, not all surfactants will be effective in the methods and compositions described herein. Applicants have thus further provided methods for evaluating the ability of a surfactant to prevent or reduce aggregation. These methods may be used to identify other surfactants that can be used in the compositions and methods described herein.

In one aspect, the present invention provides a method for evaluating the ability of a surfactant to prevent aggregation of oocysts, sporocysts, and/or sporozoites. The method comprises adding a surfactant to a composition that comprises oocysts, sporocysts, sporozoites, or any combination thereof, at a concentration sufficient to prevent aggregation of the oocysts, sporocysts, and/or sporozoites (i.e. a test concentration). The resulting composition is then agitated, for example, by shaking manually or via an automatic shaker. As previously described, agitating the composition exposes the oocysts, sporocysts, and/or sporozoites to air or other gases, and promotes aggregation. After agitation, the composition is evaluated to determine whether any aggregation has occurred. Preferably, evaluation occurs by visually observing the composition for the presence of aggregated oocysts, sporocysts, and/or sporozoites. If there is no aggregation at an interface (e.g. composition/air interface; composition/surface interface, such as composition-container or composition-cap interface), and no clumping or sticking of the oocysts (or sporocysts or sporozoites) to each other is observed, the added surfactant is considered to be effective in preventing aggregation at the test concentration. It is noted that if a surfactant is found to be effective at preventing aggregation at one concentration, that surfactant is also effective at preventing aggregation at higher concentrations.

The exact concentration of surfactant added is not critical, so long as the concentration is sufficient to prevent aggregation of the oocysts, sporocysts, and/or sporozoites. It has been observed that if the concentration of surfactant is too low, there is no effect on the prevention or reduction of aggregation, but that by raising the concentration, the problem of aggregation may be resolved. In general, a surfactant concentration of about 2.0 mg/ml or higher is sufficient for this purpose. It is noted that lower surfactant concentrations may also be effective, so long as the concentration is above the critical micelle concentration (CMC) for the surfactant being tested. However, since the minimum effective surfactant concentration may vary depending on the surfactant, a higher test concentration is preferably used.

Although not necessary to evaluate the efficacy of the surfactant, the results obtained from this test may be compared to an evaluation of a control composition, to ensure accuracy. In this aspect, the control composition (i.e. a sample of the oocyst, sporocyst, or sporozoite containing composition described above, taken prior to the addition of the surfactant being tested) is subject to the same procedure, as described above, except no surfactant is added to the composition prior to agitation. Typically, such agitation, in the absence of a surfactant, will result in some degree of oocyst, sporocyst, and/or sporozoite aggregation. Thus, if the surfactant is able to prevent aggregation in the test composition, while aggregation is observed in the control composition, the effectiveness of the surfactant at preventing aggregation of oocysts, sporocysts, and/or sporozoites is confirmed.

The results obtained in the test composition may also be compared to a second control composition which may comprise, for example, a sample of the oocyst, sporocyst, or sporozoite containing composition described above (taken prior to the addition of the surfactant to be tested), and a surfactant of known efficacy at either the test concentration, or alternatively, at a different concentration. The second control composition is then agitated and evaluated, as described above. If aggregation is observed in the test composition, while no aggregation is observed in the second control composition, the ineffectiveness of the tested surfactant at preventing the aggregation of oocysts, sporocysts, and/or sporozoites is confirmed.

In another aspect, the present invention provides a method for evaluating the ability of a surfactant to reduce oocyst, sporocyst, and/or sporozoite aggregation. In this method, a surfactant is added to a composition comprising oocysts, sporocysts, sporozoites, or any combination thereof, wherein some oocyst, sporocyst, and/or sporozoite aggregation has already occurred. The surfactant is preferably added at a concentration sufficient to reduce aggregation of the oocysts, sporocysts, and/or sporozoites. For this purpose, the surfactant concentration is preferably a test concentration, such as the test concentration described above. Upon addition of the surfactant, the resulting composition is agitated, as previously described, and then evaluated to determine whether the aggregation has been reduced or eliminated entirely by addition of the surfactant. Preferably, evaluation occurs by visually observing the composition for the presence of aggregated oocysts, sporocysts, and/or sporozoites. If no aggregation is observed (e.g. no aggregation at an interface, as described above, and there is no clumping or sticking of the oocysts, sporocysts, and/or sporozoites to each other), the added surfactant is considered to be effective at reducing aggregation at the test concentration.

Although not necessary to evaluate the efficacy of the tested surfactant, the results obtained from this test may be compared to an evaluation of one or more control composition. For example, one control composition may comprise a sample of the oocyst, sporocyst, or sporozoite containing composition described above (taken prior to the addition of the surfactant being tested), wherein some oocyst, sporocyst, and/or sporozoite aggregation has already occurred. The composition is then subjected to the same procedure, as described above, except no surfactant is added prior to agitation.

The results obtained in the test composition may also be compared to a second control, wherein a surfactant of known efficacy is added to a sample of the oocyst, sporocyst, or sporozoite containing composition (taken prior to the addition of the surfactant being tested), and wherein some oocyst, sporocyst, and/or sporozoite aggregation has already occurred. The surfactant of known efficacy may be added at the test concentration, or alternatively, at a different concentration. This second control is then agitated and evaluated, as described above.

By comparing the results obtained from the test composition to the results obtained in one or both of the above described controls, the accuracy of the test results may be confirmed.

It is noted that, in general, a surfactant found to be effective at preventing the aggregation of oocysts, sporocysts, and/or sporozoites is also effective at reducing aggregation. Likewise, a surfactant found to be effective at reducing aggregation of oocysts, sporocysts, and/or sporozoites is also effective at preventing aggregation. Furthermore, the ability of a surfactant to reduce and prevent aggregation is not dependent on the species of protozoa. Thus, a surfactant effective at preventing aggregation of one species is also effective at preventing and reducing aggregation wherein the oocysts, sporocysts, and/or sporozoites are of a plurality of species. For example, the oocysts, sporocysts, and/or sporozoites may comprise at least one species of Eimeria. As previously indicated, species of Eimeria include Eimeria acervulina, Eimeria maxima, Eimeria mitis, Eimeria tenella, Eimeria necatrix, Eimeria brunetti, and Eimeria praecox. Furthermore, the oocysts, sporocysts, and/or sporozoites may comprise a plurality of species of Eimeria. Preferably, the plurality of species comprises Eimeria acervulina, Eimeria maxima, and Eimeria tenella.

As previously discussed, surfactants may also be used in connection with the compositions described herein, and to reduce and prevent oocyst, sporocyst, and/or sporozoite aggregation in a vaccine. In general, there is no upper limit on the concentration of surfactant that is effective at reducing and preventing aggregation. However, as a practical matter, when the oocyst, sporocyst, or sporozoite containing composition is to be administered to an animal, it is preferred that the maximum surfactant concentration is not so great so as to adversely affect the health and/or performance of the animal. When used in combination with a composition to be administered to an animal, it is preferable that the concentration of surfactant be between about 0.05 mg/ml to about 10.0 mg/ml, and more preferably between about 0.05 mg/ml and about 2.0 mg/ml. When the surfactant is Tween 20 or Tween 80, it is further preferable that the surfactant concentration be between about 0.1 mg/ml and about 2.0 mg/ml.

It is noted that the minimum surfactant concentration given herein is not intended to be an absolute minimum. As previously indicated, the minimum effective surfactant concentration may vary depending on the surfactant used, and may thus be lower than these exemplary concentrations. The minimum effective surfactant concentration for the surfactants used herein may be estimated, for example, by evaluating test samples comprising progressively lower surfactant concentrations. The method described below is one example of a method for estimating the minimum effective surfactant concentration. Furthermore, when the composition comprises sporocysts or sporozoites, the minimum effective surfactant concentration is generally slightly higher than the absolute minimum concentration for oocysts, but should typically fall within the ranges given herein. The minimum effective surfactant concentration for sporocysts and sporozoites may also be estimated by the method described below.

When formulating a vaccine, it is generally preferable to include as little extraneous material as possible. As such, it may be desirable to know the specific minimum effective surfactant concentration that must be added to the vaccine to prevent or reduce oocyst, sporocyst, and/or sporozoite aggregation. This is especially advantageous since the minimum effective surfactant concentration may vary depending on the surfactant used. Methods for estimating an effective surfactant concentration are described in the Examples. Briefly, a different concentration of a surfactant of known efficacy is added to each of several tubes or vials comprising a composition containing oocysts, sporocysts, sporozoites, or any combination thereof. The resulting compositions are agitated, as described above, and visually observed for the presence of oocyst, sporocyst, and/or sporozoite aggregation. The presence of aggregation indicates that the concentration used is too low. By testing various concentrations, one skilled in the art can readily estimate the effective minimum surfactant concentration for that surfactant.

The surfactants used in connection with the compositions and methods of the present invention are preferably safe, and should not adversely affect the health or performance of a vaccinated animal. Furthermore, treatment of oocysts with the surfactants described herein does not damage the oocysts, or affect the viability or efficacy of the treated oocysts. The surfactants may be used in combination with a variety of vaccine types, as described above, including those administered by any known method, such as in ovo, oral, and intra-yolk sac injection. Furthermore, the surfactants used in the compositions and methods described herein are preferably effective at preventing or reducing the aggregation of protozoa of a single species, as well as a mixture of different species. Preferably, the surfactant is effective at preventing and reducing oocyst, sporocyst, and/or sporozoite aggregation, wherein the oocysts, sporocysts, and/or sporozoites comprise a plurality of protozoan species selected from the group consisting of E. maxima, E. acervulina, and E. tenella.

To prevent aggregation in a vaccine composition comprising oocysts, sporocysts, and/or sporozoites, it is preferable to add the surfactant to the vaccine composition during the final vaccine formulation. In other words, it is preferable to add the surfactant at the point when the vaccine composition is transferred to a container or vial that will ultimately become the final vaccine product. Alternatively, if a surfactant solution is used, the surfactant may be added to the oocysts, sporocysts, and/or sporozoites at any point during vaccine production in which it is desirable to prevent or reduce aggregation.

The surfactants described herein may also be used to reduce oocyst, sporocyst, and/or sporozoite aggregation in a vaccine composition in which aggregation has occurred. As previously discussed, oocysts, sporocysts, and/or sporozoites may aggregate at an interface, on the vaccine container's cap or stopper, or may clump to each other in the vaccine, resulting in a non-uniform distribution of oocysts, sporocysts, and/or sporozoites. This is particularly undesirable within a vaccine, as non-uniform distribution may result in over or under dosing of the vaccinated animal. As such, it is desirable to dislodge and break-up the oocysts, sporocysts, and/or sporozoites, and thus reduce aggregation (i.e. either partially reduce aggregation, or eliminate the aggregation entirely). Likewise, it is desirable to prevent oocyst, sporocyst, and/or sporozoite aggregation before such aggregation occurs.

Thus, the present invention provides a method for reducing oocyst, sporocyst, and/or sporozoite aggregation in a vaccine. The method comprises adding a surfactant capable of reducing oocyst, sporocyst, and/or sporozoite aggregation to a vaccine composition comprising oocysts, sporocysts, and/or sporozoites, wherein said oocysts, sporocysts, and/or sporozoites have aggregated. A surfactant, such as those described herein, is capable of reducing oocyst (or sporocyst or sporozoite) aggregation when, upon addition to a composition in which aggregation has occurred, the aggregation is either at least partially reduced, or eliminated entirely.

The surfactants described herein may also be used to prevent oocyst, sporocyst, and/or sporozoite aggregation in a vaccine, before such aggregation occurs. The method comprises adding a surfactant capable of preventing oocyst, sporocyst, and/or sporozoite accumulation to a vaccine composition comprising oocysts, sporocysts, and/or sporozoites.

Any of the vaccine compositions described herein may be used in combination with these methods. The vaccines may comprise a single species of oocysts, sporocysts, or sporozoites, or a plurality of species.

### EXAMPLES

### Example 1

### Evaluation of the Ability of Surfactants to Prevent Aggregation of Sporulated Oocysts

The ability of surfactants to prevent the aggregation of sporulated oocysts is tested using the following protocol. It is noted that this protocol may be adapted to test the ability of surfactants to prevent aggregation of sporocysts and sporozoites, in addition to sporulated oocysts.

To begin, two samples are taken of a composition comprising viable, sporulated oocysts. To the first (test) sample is added about 2 mg/ml of the surfactant being tested. No surfactant is added to the second (control) sample. Both samples are shaken manually. The samples are then visually examined for the presence of sporulated oocyst aggregation.

If no aggregation is observed in the test sample containing the surfactant, the surfactant is effective at preventing the aggregation of sporulated oocysts. The efficacy of these surfactants at preventing aggregation can be confirmed by comparing the test sample to the control, which will contain oocyst aggregation.

### Example 2

### Evaluation of the Ability of Surfactants to Reduce the Aggregation of Sporulated Oocysts

The ability of surfactants to reduce the aggregation of sporulated oocysts is tested using the following protocol. It is noted that this protocol may be adapted to test the ability of surfactants to reduce aggregation of sporocysts and sporozoites, in addition to sporulated oocysts.

To begin, a composition comprising viable, sporulated oocysts is shaken to induce aggregation of the sporulated oocysts. About 0.2 mg/ml of the surfactant being tested is then slowly added to the composition. The composition is then visually examined for the presence of sporulated oocyst aggregation.

If no aggregation is observed in the composition after addition of the surfactant, the surfactant is effective at reducing the aggregation of sporulated oocysts.

### Example 3

### Estimating an Effective Surfactant Concentration

Once a surfactant has been shown to be effective at reducing or preventing aggregation, the minimum concentration of the surfactant effective at preventing or reducing the aggregation of sporulated oocysts can be determined using the following protocol. It is noted that this protocol may be adapted to estimate the minimum concentration of surfactants effective to prevent and reduce aggregation of sporocysts and sporozoites, in addition to sporulated oocysts.

To begin, four to five samples are taken of a composition comprising viable, sporulated oocysts. Differing amounts of the surfactant being tested (typically 0.2 mg/ml and lower) are added to each sample, and the samples are shaken manually. The samples are then visually examined for the presence of sporulated oocyst aggregation. The presence of sporulated oocyst aggregation indicates that the concentration of surfactant used is too low.

Tween-20, Tween-80, Triton X-100, Triton X-200, Tergitol 15-S-9, and Tergitol 15-S-12 were all found to be effective at preventing the aggregation of sporulated oocysts at a concentration of 0.1 mg/ml, and above. Triton X-100, Triton X-200, Tergitol 15-S-9, and Tergitol 15-S-12 were also found to be effective at preventing sporulated oocyst aggregation at a concentration as low as 0.05 mg/ml.

### Example 4

### Oocyst Collection and Isolation

Five hundred fifty, 15 day old broiler chickens were infected with approximately 7000 viable oocysts per bird of E. tenella by oral gavage or by ingestion via drinking water or feed. Excreta y7ere collected over a three day period beginning 6 days later at 21 days of age. Total excreta collected over the three day period was 189 kg. Excreta were processed on the day collected. The excreta collected were put in a dilution tank maintained at approximately 40-50° F. and diluted with water at 0.687 to 0.630 L/bird. The diluted excreta was pumped through a 30″ diameter vibrating sieve fitted with a 50 mesh (297 micron) top screen and a 250 mesh (61 micron) bottom screen at a rate of approximately 6 LPM. The top two fractions were discarded and the filtrate, containing the oocysts, was pumped into a chilled (about 40-50° F.) collection tank and continuously agitated. The filtrate was then pumped at a feed rate of approximately 2.9-3.5 LPM into a Sharples Super-D-Canter centrifuge. The centrifuge settings were: bowl speed 3990-4004 RPM; auger speed 2306-3990 RPM and RPM delta 16.84-17.33. RPM delta is a measure of the difference between bowl and auger speeds. Total run time ranged from 97 to 100 minutes. The centrate was discarded and the solids (cake), which contained oocysts, were collected into a stainless steel tray, weighed and stored in a tank at about 40-50° F. The solids obtained from each of three collection days were combined.

The volume of solids from the combined three runs was 28 L. To these solids was added 25.6 L of high fructose corn syrup and 46.4 L of water to give a total volume of 100 L and a specific gravity of 1.094 g/l. This material was then centrifuged using a Sharples Super-D-Canter centrifuge at a bowl speed of 5998 RPM, an auger speed of 3998 RPM and a RPM delta of 20.41. The feed rate was 1.1 l/min and the total run time was 95 minutes. The centrate, containing the oocysts, was collected and stored in a tank at approximately 40-50° F. and the unwanted excreta solids discarded.

In order to remove the residual sugar in solution and to concentrate the oocysts further, the centrate was subjected to an additional centrifugation. To the 96 L of centrate obtained was added 114 L of water to give a final volume of 210 L. The centrifuge settings were bowl speed 6011 RPM and auger speed 4050 RPM. The initial RPM delta was 20.01 but was decreased to 15 and then 10 during the run to increase centrate flow. The centrate was discarded and the solid containing the oocysts was retained. The oocysts were placed in a sterilized container with sterile water at a preferred concentration of between about 5×10⁶/ml and about 50×10⁶/ml and held at from about 20° C. to about 8° C. until transferred to the sporulation vessel to undergo sporulation.

### Example 5

### Separation by Hydrocyclone

The manure from 400 host birds inoculated with E. maxima was collected from a one day period resulting in 45 kg of manure. This manure was diluted and sieved according to the method of Example 4 to give a filtrate volume of 270 L containing 6% solids and 4.64×10⁹ oocysts. The filtrate was then introduced to the hydrocyclone by a high pressure pump at a feed rate of approximately 2 gallons per minute and at a pressure of 126 psi.

The first run resulted in the an upper outlet volume (overflow) of 182 L containing 1.58×10⁸ oocysts and a lower outlet volume of 88 L containing 8% solids and 4.02×10⁹ oocysts. Overflow material was discarded after each run. A second run resulted in an upper outlet volume of 56 L containing 1.46×10⁸ oocysts and a lower outlet volume of 28 L containing a 13% solids and 3.25×10⁹ oocysts. A third and final run resulted in an overflow volume of 18 L containing 1.27×10⁸ and a final volume for the lower outlet of 10.5 L containing 27% solids and 3.56×10⁹ oocysts.

### Example 6

### Sporulation

To the oocysts obtained as described in Example 4, was added enough of a 5.25% sodium hypochlorite solution (CLOROX) to obtain a final concentration 0.05 wt % sodium hypochlorite. This oocyst/sodium hypochlorite mixture was added to a 10 liter fermentor set at 28±1° C. and an agitation rate of 200 RPM. Oxygen was provided by portable oxygen cylinders and bubbled through the mixture at a rate sufficient to obtain a percent saturation of dissolved oxygen value of at least 50% of saturation of dissolved oxygen. Oxygen flow was adjusted so as not to cause foaming of the mixture. The oocysts were maintained under these conditions for about 72 hours. During sporulation, dissolved oxygen and pH were constantly monitored. It was observed that beginning at approximately 12 hours into the sporulation process there was a decrease in the percent saturation of dissolved oxygen (increased oxygen consumption) followed by an increase in pH and a return of dissolved oxygen to previous levels (FIG. 1). In some, but not all cases, the increase in pH was preceded by a decrease in pH at about the same time as the decrease in the percent saturation of dissolved oxygen (FIG. 2). These changes in dissolved oxygen and pH were found to be reliable indicators of sporulation. Examination of oocysts following these change showed a high degree of sporulation. In contrast when these changes were not observed, the sporulation rate was dramatically reduced from approximately 90% to approximately 10%. Although sporulation was complete at approximately 24 to 36 hours, the incubation was continued for another 36 to 48 hours to provide a more stable sporulated oocyst population.

Oocyst quantities for individual species are approximately as follows:

TABLE 2

| Spp. of Eimeria | Quantity |
|---|---|
| E. acervulina | 22 million/mL |
| E. maxima | 13.6 million/mL |
| E. tenella | 13.7 million/mL |

The average oocyst sporulation ratio determined for individual species was as follows:

TABLE 3

| Spp. | Percent average |
|---|---|
| of Eimeria | sporulation ratio |
| E. acervulina | 80% |
| E. maxima | 90% |
| E. tenella | 90% |

The average oocyst viability determination for individual species was as follows:

TABLE 4

| Spp. | Average oocyst |
|---|---|
| of Eimeria | viability |
| E. acervulina | 80% |
| E. maxima | 70-80% |
| E. tenella | 80% |

### Example 7

### Tangential Flow Filtration/Sterilization

Following sporulation, the sporulated oocysts were concentrated by tangential flow filtration. To begin, the integrity of the filter membrane was visually observed prior to assembling the CONSEP system. The filter unit was then assembled according to the appropriate standard operating procedure (“SOP”) as provided in the manufacture's manual. After the system was assembled about 2 to 4 liters of cold domestic water was run through the system to check for leaks. If leaks were found, the system would be disassembled and reassembled after checking for the source of the leakage. Particular attention was paid to inspecting the gaskets to assure lack of damage to the gaskets and for proper seating.

The concentrated sporulated oocysts medium was then placed into the retentate vessel of the filter unit. Domestic cold water was added to adjust the retentate vessel volume to the desired operating level and also maintain less than a desired amount of solids. The water source was then connected to the retentate vessel through an air-tight fitting. This facilitates operating diafiltration at a constant volume.

The permeate flow valve was then closed. The control of the diaphragm pump was set to give the desired flow and then the pump was started. The permeate flow valve was then opened after substantially all of the bubbles were removed from the membranes and a steady flow was established across the membrane. The permeate was then directed to a separate collection container. The permeate sample was collected after about 2 to 5 minutes of operation and checked for sporulated oocysts. The sporulated oocysts are to be retained in the system. If sporulated oocysts were found in the permeate, the filtration would have been stopped and the source of retentate leakage identified. Retentate leakage often occurs from gaskets around the membrane or when the integrity of the membrane is compromised. The source of a leak must be detected and corrected before proceeding with permeation. Permeate collection, found to be without sporulated oocysts, was then discarded. If sporulated oocysts were found, the permeate would have been returned to the filter unit to recover any sporulated oocysts that may have leaked through into the permeate.

The flow rate of the permeate was checked periodically by measuring the volume of permeate by collecting the permeate in a graduated cylinder. The retentate tank volume was maintained at a constant volume. A small sample of the permeate was collected after every 2 liters of permeate was collected from the permeate line and the optical density at 600 nm (OD₆₀₀) was measured. Once the OD₆₀₀ of the permeate was less than 0.5, the diafiltration was stopped by closing the permeate value and disconnecting the water source. The pump's inlet lines were then removed from the retentate vessel and connected to a clean water source. The membrane were then flushed with about 500 to about 1000 ml of water to recover any sporulated oocysts. The retentate vessel was then stored overnight at about 4° C. in a refrigerator. The retentate was stored overnight to allow the sporulated oocysts to settle to the bottom of the retentate vessel. The layer of retentate over the settled oocysts was then siphoned off.

The sporulated oocysts were then sterilized. The filter unit was sterilized by using 5.25% sodium hypochlorite solution to disinfect the system. After the sodium hypochlorite was added, all subsequent procedures were conducted in a HEPA filtered laminar flow hood to maintain asepsis.

The Optisep filter unit was assembled according to the manufacture's directions. For E. maxima and E. tenella a 10-micron Spectra/Mesh filter was used. For E. acervulina a 5-micron or a 10-micron filter was used. With all species the following procedures were the same.

The inlet (retentate return) and outlet tubing (permeate) were placed in a beaker containing approximately 400 ml of about 5.25% sodium hypochlorite. The pump on the filter unit was then started to flush the system with the 5.25% sodium hypochlorite. The pump was then stopped and all the valves were closed to let the system equilibrate with the 5.25% sodium hypochlorite in the chamber and tubing for about 15 minutes.

The sporulated oocyst containing vessel was then removed from refrigeration. The supernatant was pumped out without disturbing the sporulated oocyst layer. Enough supernatant was left behind so that the total solids were less than approximately 15% by volume. The sporulated oocyst suspension was then transferred to a retentate vessel.

An equal volume of about 10% sodium hypochlorite was added to the sporulated oocyst suspension to result in a final concentration of sodium hypochlorite of approximately 5% and a solids concentration of less than about 7.5% solids in suspension. The medium was mixed thoroughly and allowed to stand for about 15 minutes.

The filtration was then begun. Autoclaved water was used as the water source for filtration. The retentate pump was activated and set at 0.6 liters per minute. The permeate line was pinched closed at this time. Once air bubbles were worked through the filter membrane and tubing the permeate line was opened and directed to a collection vessel outside the laminar flow hood. The retentate flow was then increased to 2 liters per minute. A sample of the permeate was then taken and sampled for sporulated oocysts. Finding no sporulated oocysts it was determined that there was no breach of a membrane or failure of a gasket.

The volume of the retentate vessel was maintained substantially constant by the addition of either autoclaved or sterilized water. The filtration was continued until there was no chlorine odor emanating from the permeate. This required about 10 volumes of retentate to run through the system. A sample of the permeate was then analyzed for residual chlorine. The filtration was run until the permeate sample contained less than about 1 ppm of chlorine.

Once the chlorine level was sufficiently reduced, the retentate volume was then reduced by discontinuing the addition of autoclaved or sterilized water. The concentrated retentate was then aseptically transferred to a glass vessel wherein an equal volume of 1×PBS with 60 μg/ml of gentamicin was added to the disinfected sporulated oocysts suspension. This resulted in a suspension of sporulated oocysts in 0.5×PBS with approximately 30 82 g/ml gentamicin. The solution was then placed in refrigeration at approximately 4° C. for future use.

### Example 8

### Storage

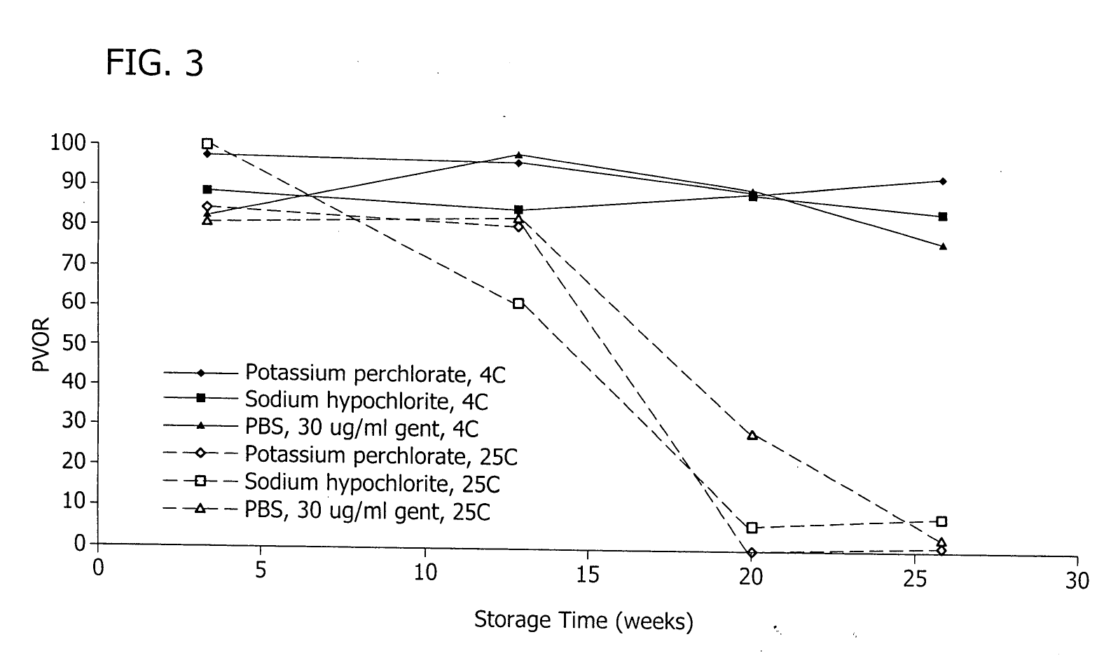

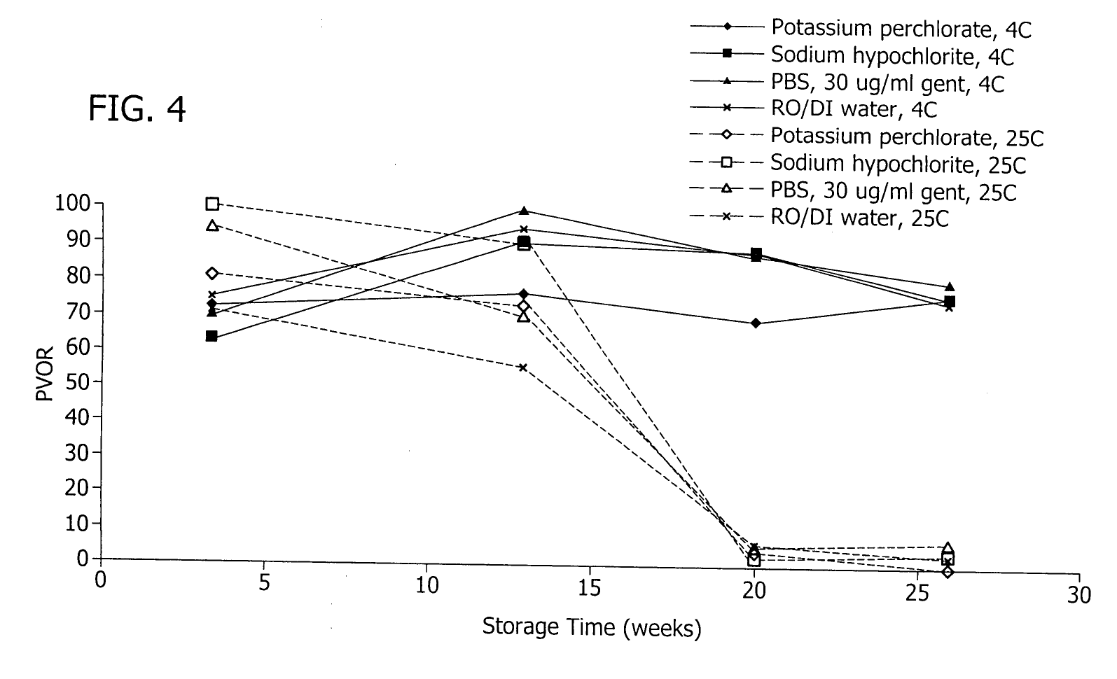

Sterile sporulated oocysts were stored in 0.1% potassium perchlorate, 0.001% sodium hypochlorite, reverse osmosis/deionized (RO/DI) water or 0.5×PBS containing 30 μg/ml gentamicin and at either 4° C. or room temperature (25° C.). In some instances the storage medium also contained Propionibacterium acnes at a concentration of 10-100 μg/dose (dry weight). As used herein, a dose is the amount to be administered to an individual animal at one time. At the times indicated in FIGS. 3 and 4, samples were aseptically removed from the storage containers and tested for viability by vital staining.

The results are shown in FIGS. 3 and 4. The method of sterilization, either 2% or 5% sodium hypochlorite, did not appear to have a significant effect on viability during storage. Sporulated oocysts which had not been sterilized, however, showed a rapid decrease in viability when stored.

Storage temperature was found to have an effect on the ability of sporulated oocysts to remain viable during storage. Sporulated oocysts maintained at 4° C. maintained their viability for at least 26 weeks when stored in any of the media tested. Sporulated oocysts stored at room temperature, however, showed a marked decrease in viability by 20 weeks in storage. When stored at 4° C., all groups of sporulated oocysts maintained at least 70% viability over the 26 week test period. In terms of change in percent viable oocysts recovered (PVOR), a comparison of PVOR at the first and last sampling periods shows that in no case did the decrease in PVOR exceed 10% when stored at 4° C. These results show that it is possible to maintain sporulated oocysts for extended periods of time in sterile medium lacking potassium dichromate without a significant loss in viability.

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A composition comprising viable sporulated oocysts of at least one species of protozoa known to cause coccidiosis, a pharmaceutically acceptable carrier, diluent, or excipient, and at least one surfactant capable of preventing or reducing the aggregation of sporulated oocysts, wherein the composition is sterile.

2. The composition of claim 1 wherein said protozoa are of the genus Eimeria.

3. The composition of claim 2 wherein said protozoa are selected from the group consisting of Eimeria acervulina, Eimeria maxima, Eimeria mitis, Eimeria tenella, Eimeria necatrix, Eimeria brunetti, Eimeria praecox, and combinations thereof.

4. The composition of claim 1 wherein said protozoa comprise a plurality of species of protozoa.

5. The composition of claim 4 wherein said plurality of species comprise Eimeria acervulina, Eimeria maxima, and Eimeria tenella.

6. The composition of claim 1 wherein said surfactant is selected from the group consisting of anionic surfactants, non-ionic surfactants, and combinations thereof.

7. The composition of claim 6 wherein said surfactant is a non-ionic surfactant selected from the group consisting of Tween 20, Tween 80, Triton X-100, Triton X-200, Tergitol 15-S-9, Tergitol 15-S-12, and combinations thereof.

8. The composition of claim 1 wherein said surfactant is present in a concentration of from about 0.05 mg/ml to about 10.0 mg/ml.

9. The composition of claim 8 wherein said surfactant is present in a concentration of from about 0.05 mg/ml to about 2.0 mg/ml.

10. The composition of claim 8 wherein said surfactant is present in a concentration of from about 0.1 mg/ml to about 2.0 mg/ml.

11. The composition of claim 1 wherein said aggregation is at an interface.

12. The composition of claim 11 wherein said interface is selected from the group consisting of a composition-air interface, a composition-container interface, or any combination thereof.

13. The composition of claim 1 wherein said aggregation is on a container cap or stopper.

14. The composition of claim 1 wherein said composition comprises one or more dosage unit.

15. The composition of claim 14 wherein each dosage unit comprises not more than about 10× the minimum immunizing dose of said oocysts.

16. The composition of claim 1 wherein said composition is substantially free of bacterial contamination.

17. The composition of claim 16 wherein said bacterial contamination is removed by tangential flow filtration.

18. The composition of claim 1 wherein bacterial contaminants have been removed from said composition at one or more step(s) of production.

19. The composition of claim 18 wherein said contaminants are removed by tangential flow filtration.

20. The composition of claim 1 wherein said composition further comprises:
not more than about 0.8% by weight of alkali metal dichromate;
not more than about 0.75% chloramine by weight;
not more than about 10.0 ppm hypochlorite ion; and
not more than about 1000 mg/l hydrogen peroxide.

21. The composition of claim 1 wherein the diluent comprises water.

22. The composition of claim 21 wherein the aqueous diluent comprises 0.5× phosphate buffered saline.

23. The composition of claim 22 further comprising gentamicin.

24. The composition of claim 23 wherein said gentamicin is present in an amount of about 30 μg/ml.

25. A preparation for the prevention and treatment of coccidiosis comprising:
a pharmaceutically acceptable carrier, diluent, or excipient;
live sporulated oocysts of at least one species of coccidial protozoa; and
an amino acid;
wherein the sporulated oocysts are sanitized.

26. A preparation for the prevention and treatment of coccidiosis comprising:
a pharmaceutically acceptable carrier, diluent, or excipient;
live sporulated oocysts of at least one species of coccidial protozoa; and
a surfactant;
wherein the sporulated oocysts are sanitized.

27. The preparation of claim 26 wherein the surfactant is selected from the group consisting of ionic detergents and non-ionic detergents.
